(19) United States
(12) Patent Application Publication
(10) Pub. No.: US 2004/0209929 A1
(43) Pub. Date: Oct. 21, 2004

(54) Fused heteroaryl carboxylic acids as PPAR agonists

(75) Inventors: Donald Skalitzky, Ann Arbor, MI (US); Wei-Guo Su, San Diego, CA (US); Luke Raymond Zehnder, San Diego, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 10/783,654

(22) Filed: Feb. 19, 2004

Related U.S. Application Data
(60) Provisional application No. 60/448,931, filed on Feb. 21, 2003.

(51) Int. Cl. C07D 417/02; C07D 413/02; C07D 403/02; A61K 31/427; A61K 31/422

(52) U.S. Cl. 514/365; 514/374; 514/394; 514/397; 548/181; 548/215; 548/306.1; 548/311.7

(57) ABSTRACT

Fused heteroarylcarboxylic acids of formula (I):

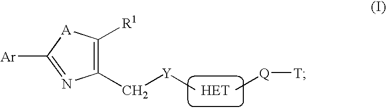

wherein R¹, Ar, A, Y, HET, Q, and T are as defined in the specification; pharmaceutical compositions containing effective amounts of said compounds or their salts are useful for treating PPAR related disorders, such as diabetes, dyslipidemia, obesity and inflammatory disorders.

This application claims benefit of priority from U.S. Provisional Application No. 60/448,931 filed on Feb. 21, 2003.

### FIELD OF THE INVENTION

This invention relates to fused heteroaryl carboxylic acids, specifically indazole and aza-indole carboxylic acids, that exhibit agonist activity to peroxisome proliferator-activated receptor (PPAR) enabling them to be useful in modulation of blood glucose and the increase of insulin sensitivity in mammals. This invention also relates to treatment of PPAR related disorders, such as diabetes, dyslipidemia, obesity and inflammatory disorders.

### BACKGROUND OF THE INVENTION

Peroxisome proliferators are a structurally diverse group of compounds which, when administered to rodents, elicit dramatic increases in the size and number of hepatic and renal peroxisomes, as well as concomitant increases in the capacity of peroxisomes to metabolize fatty acids via increased expression of the enzymes required for the β-oxidation cycle. Chemicals included in this group are the fibrate class of hypolipidermic drugs, herbicides, and phthalate plasticizers (Reddy and Lalwani, Crit. Rev. Toxicol, 12:1-58 (1983)). Peroxisome proliferation can also be elicited by dietary or physiological factors such as a high-fat diet and cold acclimatization.

Insight into the mechanism whereby peroxisome proliferators exert their pleiotropic effects was provided by the identification of a member of the nuclear hormone receptor superfamily activated by these chemicals (Isseman and Green, Nature, 347-645-650 (1990)). This receptor, termed peroxisome proliferator activated receptor alpha (PPAR-α), was subsequently shown to be activated by a variety of medium and long-chain fatty acids and to stimulate expression of the genes encoding rat acyl-CoA oxidase and hydratase-dehydrogenase (enzymes required for peroxisomal β-oxidation), as well as rabbit cytochrome P450 4A6, a fatty acid Ω-hydroxylase. PPAR-α activates transcription by binding to DNA sequence elements, termed peroxisome proliferator response elements (PPRE), as a heterodimer with the retinoid X receptor. The retinoid X receptor is activated by 9-cis retinoic acid (see Kliewer, et al., Nature, 358:771-774 (1992), Gearing, et al., Proc. Natl. Acad. Sci., USA 90:1440-1444 (1993), Keller, et al., Proc. Natl. Acad. Sci. USA, 90:2160-2164 (1993), Heyman, et al., Cell, 68:397-406 (1992), and Levin, et al., Nature, 355:359-361 (1992)). Since the PPAR-α-RXR complex can be activated by peroxisome proliferators and/or 9-cis retinoic acid, the retinoid and fatty acid signaling pathways are seen to converge in modulating lipid metabolism. Since the discovery of PPAR-α, additional isoforms of PPAR have been identified, e.g., PPAR-β, PPAR and PPAR-δ, which are spatially differentially expressed. Each PPAR receptor shows a different pattern of tissue expression, and differences in activation by structurally diverse compounds. PPAR-γ, for instance, is expressed most abundantly in adipose tissue and at lower levels in skeletal muscle, heart, liver, intestine, kidney, vascular endothelial and smooth muscle cells as well as macrophages. Two isoforms of PPAR-γ exist, identified as γ₁ and γ₂, respectively. PPAR-γ mediates adipocyte signalling, lipid storage, and fat metabolism. Evidence gathered to date support the conclusion that PPAR-γ is the primary, and perhaps the only, molecular target mediating the insulin sensitizing action of one class of antidiabetic agents, the thiazolidine 2,4 diones.

In a monotherapeutic or combination therapy context, new and established oral antidiabetic agents are still considered to have non-uniform and even limited effectiveness. The effectiveness of oral antidiabetic therapies may be limited, in part, because of poor or limited glycemic control, or poor patient compliance due to unacceptable side effects. These side effects include edema weight gain, or even more serious complications. For instance, hypoglycemia is observed in some patients taking sulfonylureas. Mefformin, a substituted biguanide, can cause diarrhea and gastrointestinal discomfort. Finally, edema, weight gain, and in some cases, hepatoxicity, have been linked to the administration of some thiazolidine 2,4 dione antidiabetic agents. Combination therapy using two or more of the above agents is common, but generally only leads to incremental improvements in glycemic control.

As a result, there is a need for oral antidiabetic agents that can be used alone or in combination, and that do not give rise to side effects such as fluid retention, peripheral edema, weight gain, or more severe complications.

Because there are several isoforms of PPAR, it would also be desirable to identify compounds that are capable of selectively interacting with only one of the PPAR isoforms, preferably PPAR-γ. Such compounds may modulate processes mediated by PPAR, preferably PPAR-α and PPAR-γ, such as, for example, diabetes, dyslipidemia, obesity and inflammatory disorders, and the metabolic syndrome (i.e., impaired glucose tolerance, insulin resistancem hyoertriglyceridemia and/or obesity).

### SUMMARY OF THE INVENTION

The present invention provides novel compounds of Formula (I):

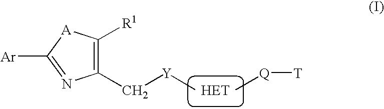

wherein:

Ar is (C₃-C₁₀)cycloalkyl, (C₂-C₁₀)heterocyclyl, (C₆-C₁₀)aryl, or (C₁-C₁₀)heteroaryl, wherein each Ar is optionally substituted with one to four substituents selected from Z;

A is —CH₂—, —NH, —O—, or —S—;

R¹ is (C₁-C₈)alkyl, (C₃-C₁₀)cycloalkyl, (C₂-C₁₀)heterocyclyl, (C₆-C₁₀)aryl, or (C₁-C₁₀)heteroaryl; wherein each R¹ is optionally substituted with one to four substituents selected from Z¹;

Y is selected from the group consisting of —(CH₂)ₙ—, —(CH₂)ₙ—NR¹⁵—, —(CH₂)ₙ—O—, and —(CH₂)ₙ—S—; wherein each n is independently 0, 1, 2, or 3;

and R¹⁵ is hydrogen, (C₁-C₈)alkyl, (C₃-C₁₀)cycloalkyl, (C₂-C₁₀)heterocyclyl, (C₆-C₁₀)aryl, or (C₁-C₁₀)heteroaryl; wherein each R¹⁵ is optionally substituted with one to four substituents selected from Z²;

Q is selected from the group consisting of —(CR²R³)ₘ—, —(CR²R³)ₘ—N¹⁵—, —N¹⁵—(CR²R³)ₘ—, (CR²R³)ₘ—O—, —O—(CR²R³)ₘ—, S—(CR²R³)ₘ—, and —(CR²R³)ₘ—S—; wherein each m is independently 1, 2, 3, 4, 5, or 6;

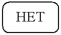

is a fused (C₆-C₁₂)heteroaryl optionally substituted one to four substituents selected from Z³, wherein Z³ may be in any ring of the fused (C₆-C₁₂)heteroaryl, having the formula:

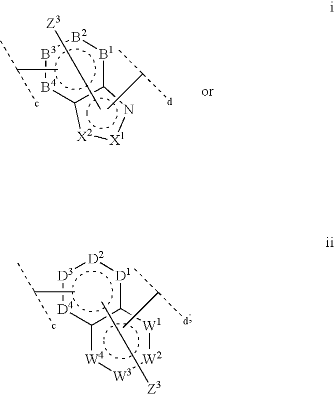

wherein the doffed lines are optional double bonds such that said fused (C₆-C₁₂)heteroaryl is aromatic;

Each of X¹, X², W¹, W², W³, W⁴, B¹, B², B³, B⁴, D¹, D², D³ and D⁴ are independently ═CH— or ═N—;

At least one of X¹, X², B¹, B², B³ and B⁴ must be ═N—;

At least one of W¹, W², W³, W⁴, D¹, D², D³ and D⁴ must be ═N—;

Wherein each --c is a point of attachment to the group —Y— and each ---d is a point of attachment to the group —Q—;

Each of Z, Z¹, Z², and Z³ are selected from the group consisting of:

(a) H, F, Cl, Br, CF₃, or CN;

(b) (C₁-C₈)alkyl optionally substituted with one to four substituents independently selected from R⁷;

(c) —C(═O)—R⁴ {wherein R⁴ is selected from the group consisting of H, OH, CF₃, (C₁-C₈)alkyl, (C₁-C₈)alkyl-O—, (C₃-C₁₀)cycloalkyl, (C₃-C₁₀)cycloalkyl-O—; (C₂-C₁₀)heterocyclyl, (C₂-C₁₀)heterocyclyl-O—; (C₆-C₁₀)aryl, (C₆-C₁₀)aryl-O—, (C₁-C₁₀)heteroaryl, and (C₁-C₁₀)heteroaryl-O—};

(d) —C(═O)—NR⁵R⁶ {wherein R⁵ is H or (C₁-C₈)alkyl; and wherein R₆ is selected from the group consisting of H, (C₁-C₈)alkyl, —CH₂—(C═O)—O—(C₁-C₈)alkyl, (C₃-C₁₀)cycloalkyl, (C₂-C₁₀)heterocyclyl, (C₆-C₁₀)aryl, and (C₁-C₁₀)heteroaryl};

(e) (C₃-C₁₀)cycloalkyl, (C₂-C₁₀)heterocyclyl, (C₆-C₁₀)aryl, or (C₁-C₁₀)heteroaryl;

(f) NR⁹R¹⁰ {wherein R⁹ is independently H or (C₁-C₈)alkyl; R¹⁰ is selected from the group consisting of —C(═O)—O—C(CH₃)₃, (C₁-C₈)alkyl, (C₃-C₁₀)cycloalkyl, (C₂-C₁₀)heterocyclyl, (C₆-C₁₀)aryl, and (C₁-C₁₀)heteroaryl; or R⁹ and R¹⁰ may optionally be taken together with the nitrogen to which they are attached to form a 5 to 8-membered heteroaryl or heterocyclyl ring};

(g) R¹¹—O— {wherein R¹¹ is selected from the group consisting of (C₁-C₈)alkyl, (C₃-C₁₀)cycloalkyl, (C₂-C₁₀)heterocyclyl, (C₆-C₁₀)aryl, and (C₁-C₁₀)heteroaryl};

(h) R¹²—SOₚ— {wherein R¹² is selected from the group consisting of (C₁-C₈)alkyl, (C₃-C₁₀)cycloalkyl, (C₂-C₁₀)heterocyclyl, (C₆-C₁₀)aryl, and (C₁-C₁₀)heteroaryl; and wherein p is 0, 1, or 2}; and

(i) R¹³R¹⁴N—SO_{q}— {wherein R¹³ is H or (C₁-C₈)alkyl; R¹⁴ is (C₁-C₈)alkyl, (C₃-C₁₀)cycloalkyl, (C₂-C₁₀)heterocyclyl, (C₆-C₁₀)aryl, or (C₁-C₁₀)heteroaryl; or R¹³ and R¹⁴ may optionally be taken together with the nitrogen to which they are attached to form a 5 to 8-membered heteroaryl or heterocyclyl ring; and wherein q is 1 or 2};

Each of R² and R³ is independently (a) H; (b) (C₁-C₈)alkyl optionally substituted with one to four substituents independently selected from R⁷; (c) COOH; or (d) (C₆-C₁₀)aryl optionally substituted with one to four substituents independently selected from R₈;

Wherein each of R⁷and R⁸ are independently selected from the group consisting of:

(a) F, Cl, Br, I, CN, CF₃, or NO₂;

(b) —NR⁹R¹⁰ {wherein R⁹ is independently H or (C₁-C₈)alkyl; R¹⁰ is selected from the group consisting of —C(═O)—O—C(CH₃)₃, (C₁-C₈)alkyl, (C₃-C₁₀)cycloalkyl, (C₂-C₁₀)heterocyclyl, (C₆-C₁₀)aryl, and (C₁-C₁₀)heteroaryl; or R⁹ and R¹⁰ may optionally be taken together with the nitrogen to which they are attached to form a 5 to 8-membered heteroaryl or heterocyclyl ring};

(c) R¹¹—O— {wherein R¹¹ is selected from the group consisting of (C₁-C₈)alkyl, (C₃-C₁₀)cycloalkyl, (C₂-C₁₀)heterocyclyl, (C₆-C₁₀)aryl, and (C₁-C₁₀)heteroaryl};

(d) R¹²-SOₚ— (wherein R¹² is selected from the group consisting of (C₁-C₈)alkyl, (C₃-C₁₀)cycloalkyl, (C₂-C₁₀)heterocyclyl, (C₆-C₁₀)aryl, and (C₁-C₁₀)heteroaryl; and wherein p is 0, 1, or 2); and

(e) R¹³R¹⁴N—SO_{q}— {wherein R¹³ is H or (C₁-C₈)alkyl; R¹⁴ is (C₁-C₈)alkyl, (C₃-C₁₀)cycloalkyl, (C₂-C₁₀)heterocyclyl, (C₆-C₁₀)aryl, or (C₁-C₁₀)heteroaryl; or R¹³ and R¹⁴ may optionally be taken together with the nitrogen to which they are attached to form a 5 to 8-membered heteroaryl or heterocyclyl ring; and wherein q is 1 or 2};

(f) T is selected from the group consisting of —(C═O)—OH, —(C═O)—OR¹⁵, —(C═O)—OM {wherein M is an alkali metal or alkaline earth metal), tetrazolyl, thiazolidinyl, —SO₂—NH—R¹⁵, —NH—SO₂—R¹⁵, —(C═O)—NH—SO₂—R¹⁵, and other acid prodrug or isosteres thereof;

or a pharmaceutically acceptable salt thereof.

In one embodiment of the present invention,

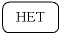

is a fused (C₆-C₁₂)heteroaryl optionally substituted one to four substituents selected from Z³, wherein Z³ may be in any ring of the fused (C₆-C₁₂)heteroaryl, having the formula:

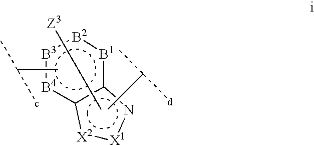

selected from the group consisting of:

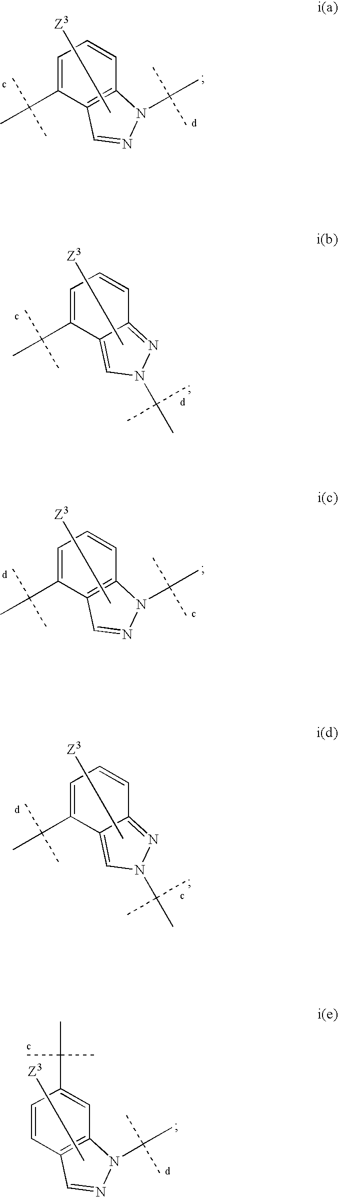

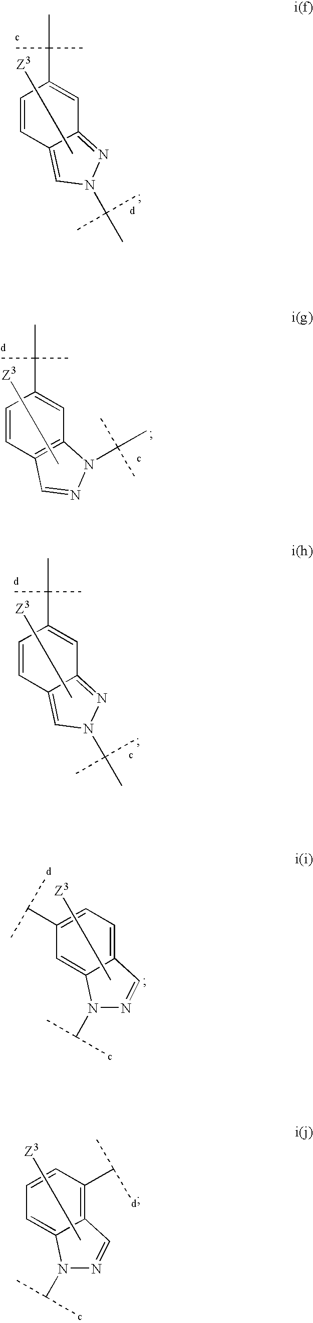

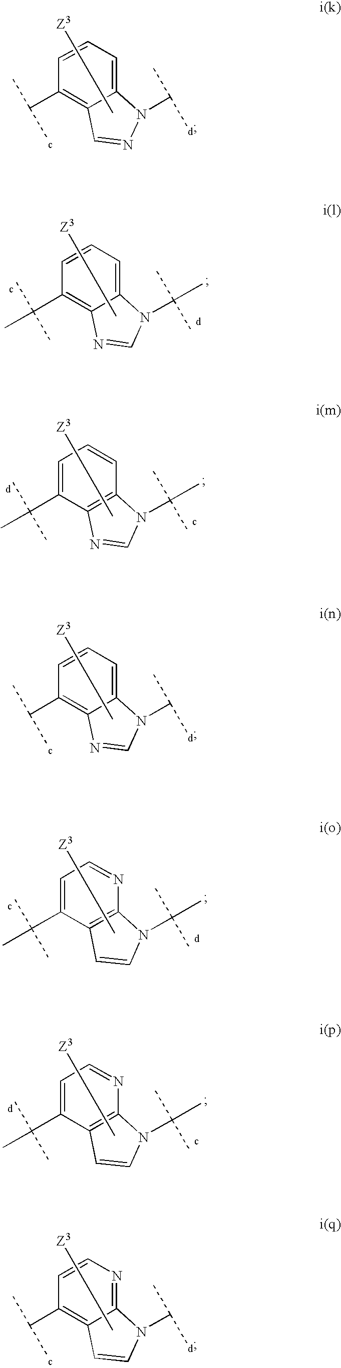

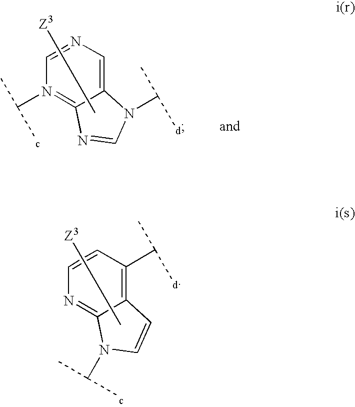

In another embodiment, the invention relates to compounds of the Formula (I) wherein

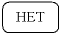

is a fused (C₆-C₁₂)heteroaryl optionally substituted one to four substituents selected from Z³, wherein Z³ may be in any ring of the fused (C₆-C₁₂)heteroaryl, having the formula (ii):

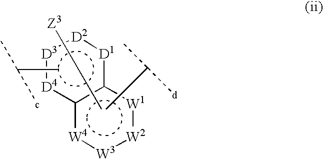

selected from the group consisting of:

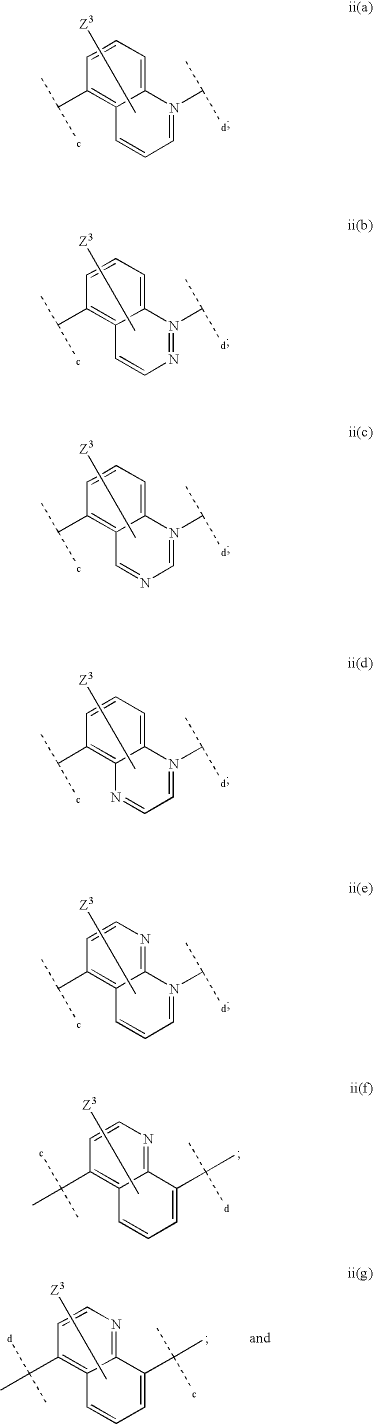

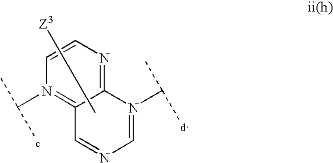

In another embodiment, the invention relates to compounds of the Formula (I) wherein Ar is phenyl.

In another preferred embodiment, the invention relates to compounds of the Formula (I) wherein A is —O—.

In another embodiment, the invention relates to compounds of the Formula (I) wherein R¹ is (C₁-C₈)alkyl, preferably methyl.

In another embodiment, the invention relates to compounds of the Formula (I) wherein R¹ is (C₆-C₁₀)aryl, preferably phenyl.

In another embodiment, the invention relates to compounds of the Formula (I) wherein Q is —(CR²R³)ₘ—, m is 2 or 3, and each of R² and R³ is hydrogen or (C₁-C₈)alkyl.

In another embodiment, the invention relates to compounds of the Formula (I) wherein Q is —(CR²R³)ₘ—NH—, m is 1 or 2, and each of R² and R³ is hydrogen or unsubstituted (C₁-C₈)alkyl.

In another embodiment, the invention relates to compounds of the Formula (I) wherein Q is —(CR²R³)ₘ—O—, m is 1 or 2, and each of R² and R³ is hydrogen or unsubstituted (C₁-C₈)alkyl.

In another embodiment, the invention relates to compounds of the Formula (I) wherein Q is —(CR²R³)ₘ—S—, m is 1 or 2, and each of R² and R³ is hydrogen or unsubstituted (C₁-C₈)alkyl.

In another preferred embodiment, the invention relates to compounds of the Formula (I) wherein T is —(C═O)—OH.

In another embodiment, the invention relates to compounds of the Formula (I) wherein T is selected from the group consisting of tetrazolyl, thiazolidinyl, —SO₂—NH—R¹⁵, —NH—SO₂—R¹⁵, —(C═O)—NH—SO₂—R¹⁵, and other acid prodrug or isosteres thereof.

In another embodiment, the invention relates to compounds of the Formula (I) wherein Z³ is selected from the group consisting of F, Cl, Br, or I.

In another embodiment, the invention relates to compounds of the Formula (I) wherein Z³ is (C₁-C₈)alkyl, preferably unsubstituted (C₁-C₈)alkyl.

In another embodiment, the invention relates to compounds of the Formula (I) wherein Y is —(CH₂)ₙ—O— and n is 1, 2, or 3.

In another embodiment, the invention relates to compounds of the Formula (I) wherein Y is —(CH₂)ₙ—NR¹⁵—, wherein R¹⁵ is hydrogen, (C₁-C₈)alkyl or (C₃-C₁₀)cycloalkyl, and n is 1, 2, or 3.

In another embodiment, the invention relates to compounds of the Formula (I) wherein Y is —(CH₂)ₙ— and n is 1, 2, or 3.

In another embodiment, the invention relates to compounds of the Formula (I) wherein Y is —(CH₂)ₙ—S— and n is 1, 2, or 3.

In another embodiment, the invention relates to compounds of the Formula (I) selected from the group consisting of:

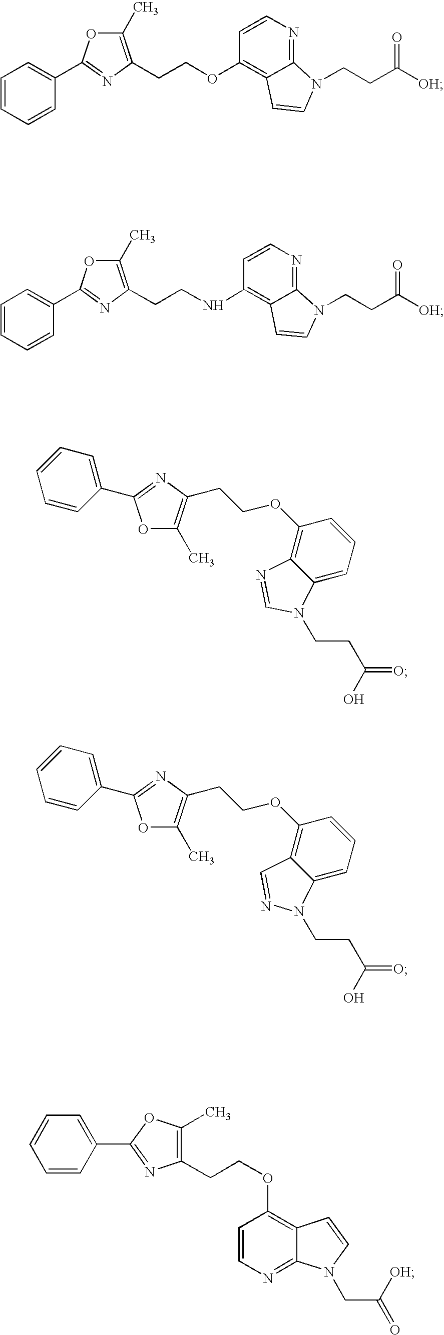

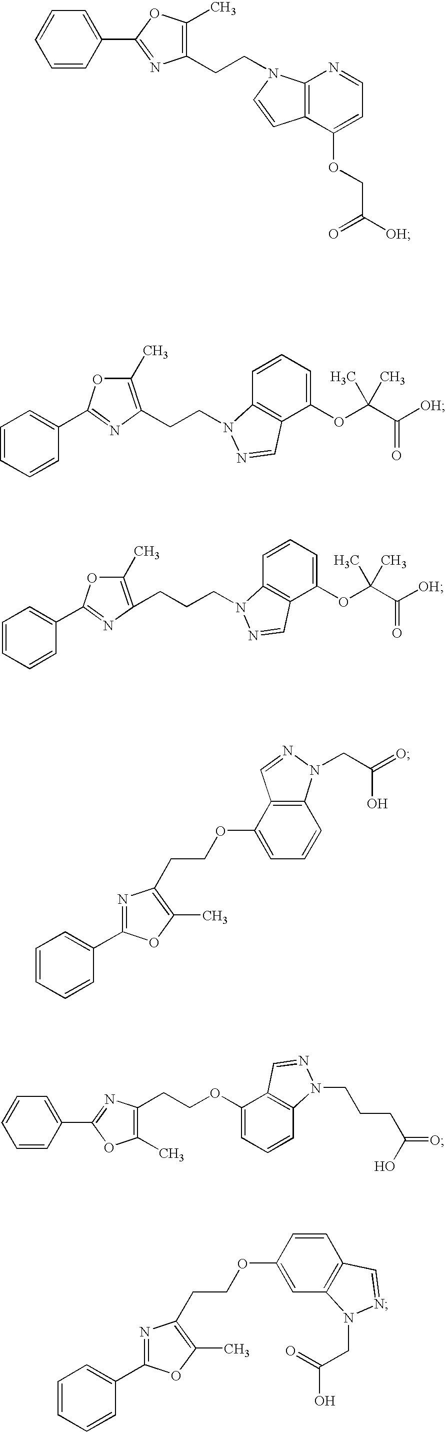

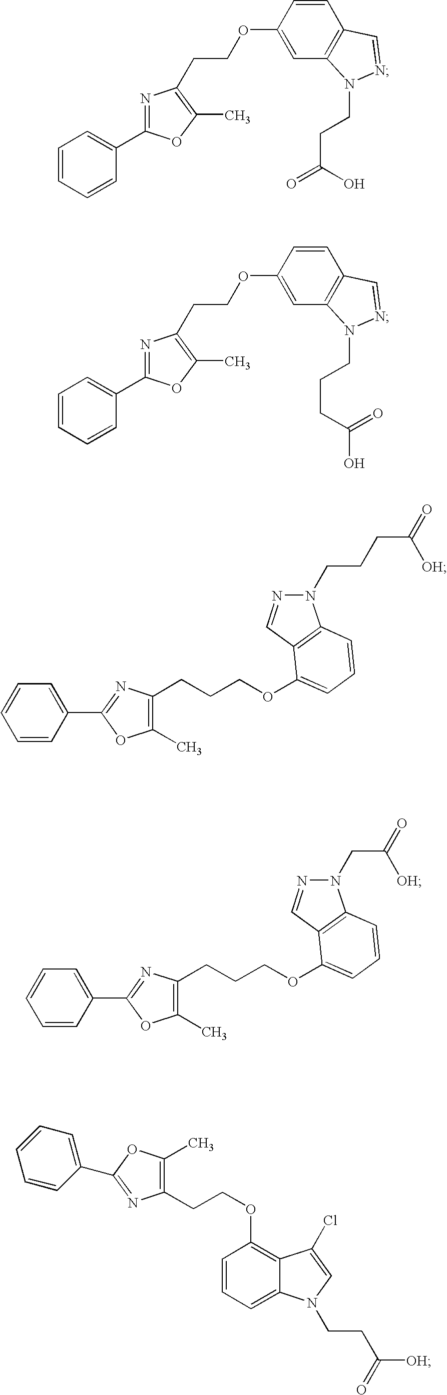

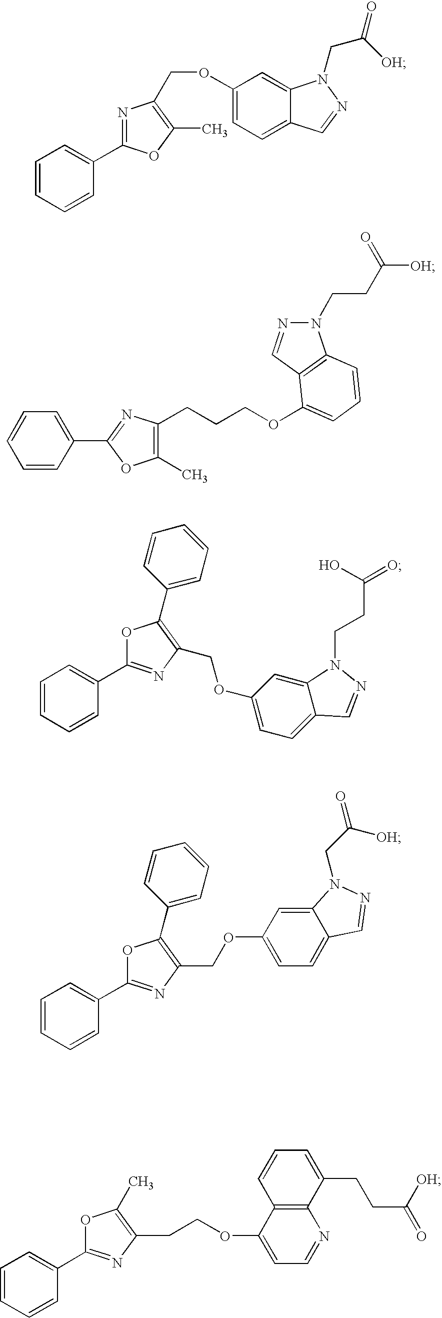

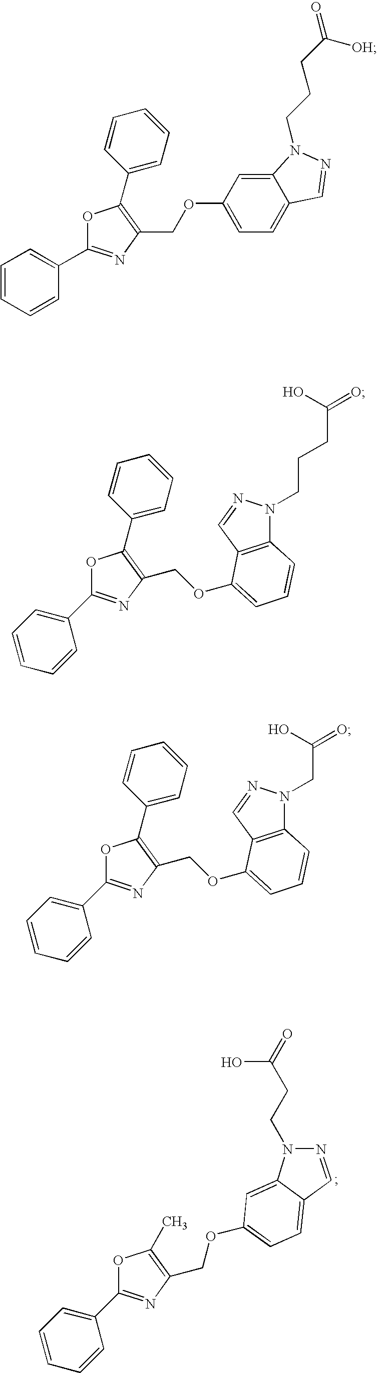

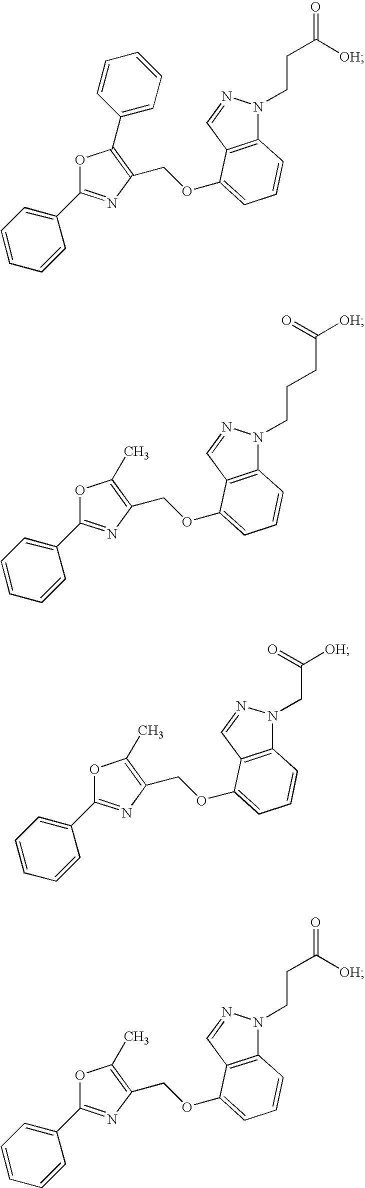

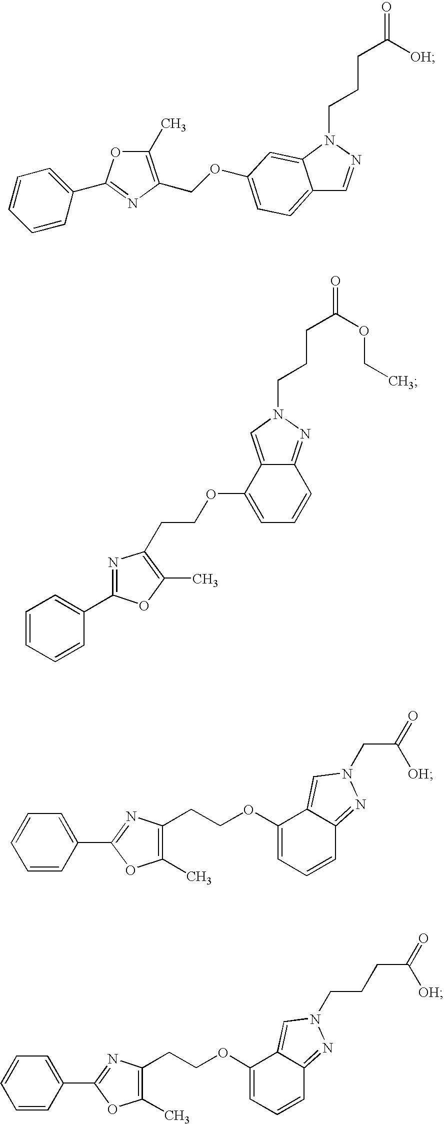

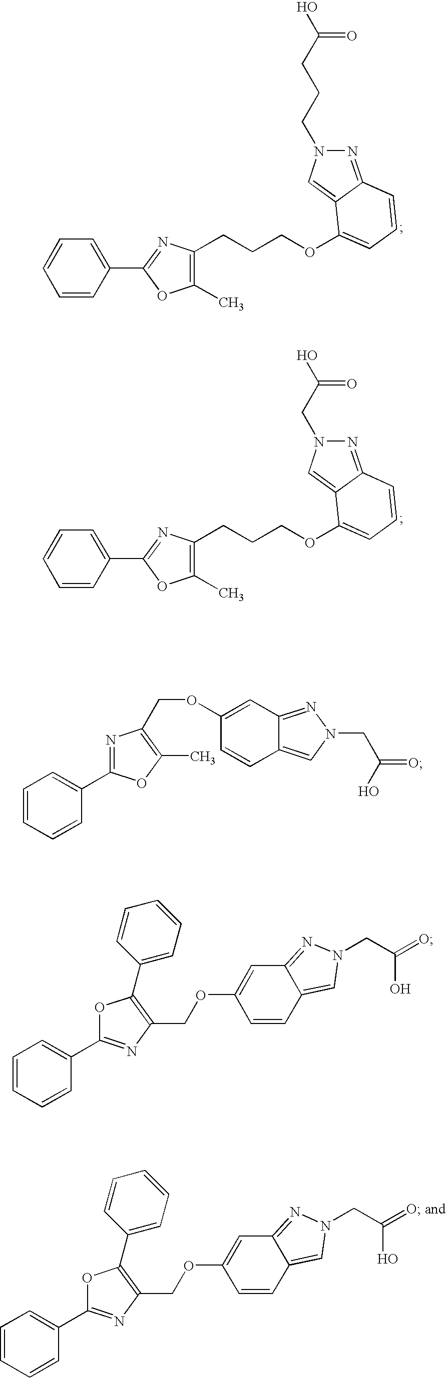

the pharmaceutically acceptable salts thereof.

In another embodiment, the invention relates to

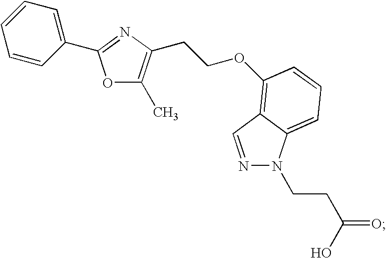

or the pharmaceutically acceptable salts thereof.

In another embodiment, the invention relates to

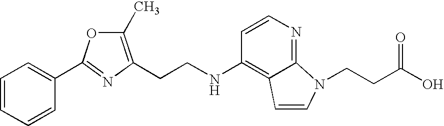

or the pharmaceutically acceptable salts thereof.

In another embodiment, the invention relates to

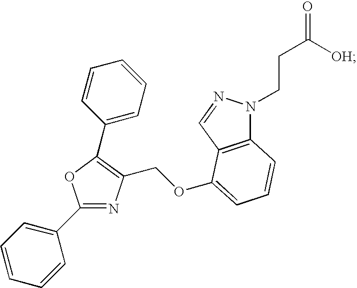

or the pharmaceutically acceptable salts thereof.

In another embodiment, the invention relates to

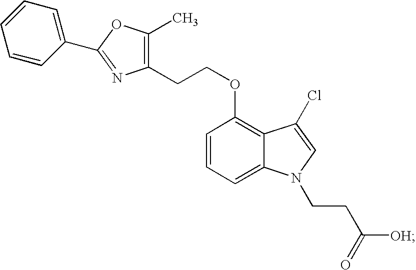

or the pharmaceutically acceptable salts thereof.

In another embodiment, the invention relates to

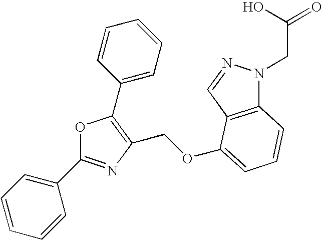

or the pharmaceutically acceptable salts thereof.

In another embodiment, the invention relates to

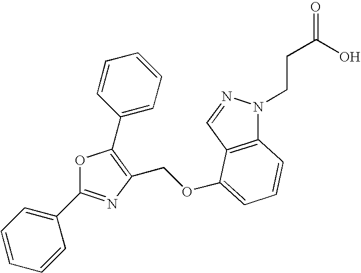

or the pharmaceutically acceptable salts thereof.

In another embodiment, the invention relates to

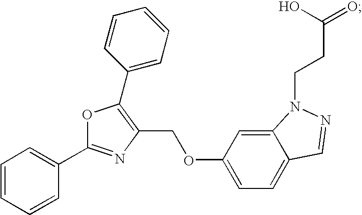

or the pharmaceutically acceptable salts thereof.

In another embodiment, the invention relates to

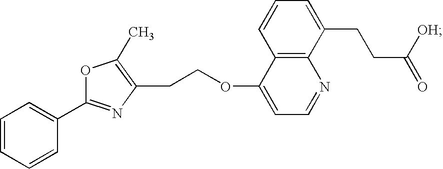

or the pharmaceutically acceptable salts thereof.

In another embodiment, the invention relates to

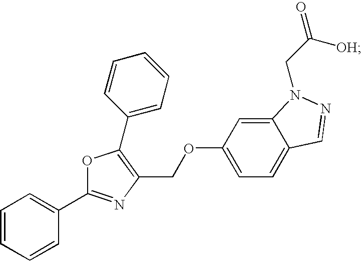

or the pharmaceutically acceptable salts thereof.

In another embodiment, the invention relates to

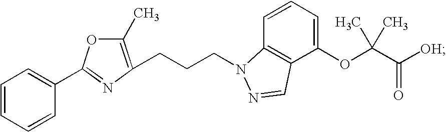

or the pharmaceutically acceptable salts thereof.

The present invention also provides a method of treating non-insulin dependent diabetes mellitus in a mammal comprising administering to the mammal in need thereof a therapeutically effective amount of a compound of Formula (I). In one embodiment, said mammal has an impaired glucose tolerance.

The present invention also provides a method of treating polycystic ovarian syndrome in a mammal comprising administering to the mammal in need thereof a therapeutically effective amount of a compound of Formula (I).

The present invention also provides a method of treating obesity in a mammal comprising administering to the mammal in need thereof a therapeutically effective amount of a compound of Formula (I).

The present invention also provides a method of reducing body weight in an obese mammal comprising administering to the mammal in need thereof a therapeutically effective amount of a compound of Formula (I).

The present invention also provides a method of treating hyperglycemia in a mammal comprising administering to the mammal in need thereof a therapeutically effective amount of a compound of Formula (I).

The present invention also provides a method of treating hyperlipidemia in a mammal comprising administering to the mammal in need thereof a therapeutically effective amount of a compound of Formula (I).

The present invention also provides a method of treating hypercholesteremia in a mammal comprising administering to the mammal in need thereof a therapeutically effective amount of a compound of Formula (I).

The present invention also provides a method of treating atherosclerosis in a mammal comprising administering to the mammal in need thereof a therapeutically effective amount of a compound of Formula (I).

The present invention also provides a method of treating hypertriglyceridemia in a mammal comprising administering to the mammal in need thereof a therapeutically effective amount of a compound of Formula (I).

The present invention also provides a method of treating hyperinsulinemia in a mammal comprising administering to the mammal in need thereof a therapeutically effective amount of a compound of Formula (I).

The present invention also provides a method of treating a patient suffering from abnormal insulin and/or evidence of glucose disorders associated with circulating glucocorticoids, growth hormone, catecholamines, glucagon, or parathyroid hormone, comprising administering to said patient a therapeutically effective amount of a compound of Formula (I).

The present invention also provides a method of treating insulin resistance syndrome in humans comprising administering to a patient in need of treatment a therapeutically effective amount of a compound of Formula (I).

The present invention also provides a method of treating PPAR-related disorders in humans comprising administering to a patient in need of treatment a therapeutically effective amount of a compound of Formula (I).

The present invention also provides a method of modulating PPAR activity in a mammal, comprising administering to a mammal a therapeutically effective amount of a compound of Formula (I).

The present invention also provides a method of lowering blood glucose in a mammal, comprising administering to a mammal an amount of a compound of Formula (I) effective to lower blood glucose levels.

The present invention also provides a method of modulating fat cell differentiation in a mammal, comprising administering to a mammal a therapeutically effective amount of a compound of Formula (I).

The present invention also provides a method of modulating processes mediated by PPAR in a mammal, comprising administering to a mammal a therapeutically effective amount of a compound of Formula (I).

The present invention also provides a method of increasing insulin sensitivity in mammals, comprising administering to a mammal a therapeutically effective amount of a compound of Formula (I).

The present invention also provides a composition comprising at least one modulator of PPAR of Formula (I) and a pharmaceutically acceptable carrier thereof. Exemplary pharmaceutically acceptable carriers include carriers suitable for oral, intravenous, subcutaneous, intramuscular, intracutaneous, and the like administration. Administration in the form of creams, lotions, tablets, dispersible powders, granules, syrups, elixirs, sterile aqueous or non-aqueous solutions, suspensions or emulsions, and the like, is contemplated.

For the preparation of oral liquids, suitable carriers include emulsions, solutions, suspensions, syrups, and the like, optionally containing additives such as wetting agents, emulsifying and suspending agents, sweetening, flavoring and perfuming agents, and the like.

For the preparation of fluids for parenteral administration, suitable carriers include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized, for example, by filtration through a bacteria-retaining filter, by incorporating sterilizing agents into the compositions, by irradiating the compositions, or by heating the compositions. They can also be manufactured in the form of sterile water, or some other sterile injectable medium immediately before use.

### Definitions

For purposes of the present invention, as described and claimed herein, the following terms are defined as follows:

Unless otherwise indicated, the term “(C₁-C₈)alkyl” as well as the (C₁-C₈)alkyl component of other terms referred to herein (e.g., the “(C₁-C₈)alkyl component of (C₁-C₈)alkyl-O—), may be linear or branched (such as methyl, ethyl, n-propyl, isopropyl, n-butyl, isobutyl, secondary-butyl, tertiary-butyl).

Unless otherwise indicated, the term “(C₃-C₁₀)cycloalkyl” refers to a non-aromatic, saturated or partially saturated, monocyclic or fused, spiro or unfused bicyclic or tricyclic hydrocarbon referred to herein containing a total of from 3 to 10 carbon atoms, preferably 5-8 ring carbon atoms. Exemplary (C₃-C₁₀)cycloalkyls include monocyclic rings having from 3-7, preferably 3-6, carbon atoms, such as cyclopropyl, cyclobutyl, cyclopentyl, cyclohexyl, cycloheptyl and the like. Illustrative examples of (C₃-C₁₀)cycloalkyl are derived from, but not limited to, the following:

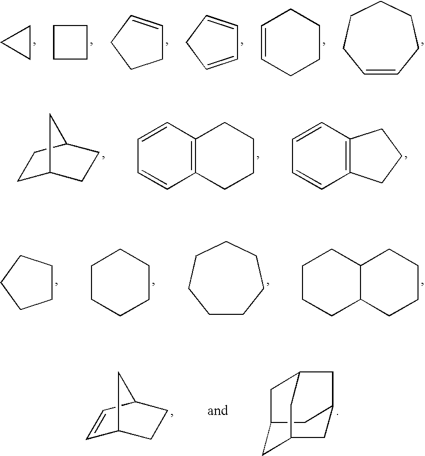

Unless otherwise indicated, the term “(C₂-C₁₀)heterocyclyl” refers to a non-aromatic, saturated or partially saturated, monovalent, monocyclic or fused, spiro or unfused bicyclic or tricyclic functional groups referred to herein containing a total of from 2 to 10 ring carbon atoms and 1 to 5 ring heteroatoms selected from nitrogen, oxygen and sulfur. Illustrative examples of (C₂-C₁₀)heterocyclyl are derived from, but not limited to, the following:

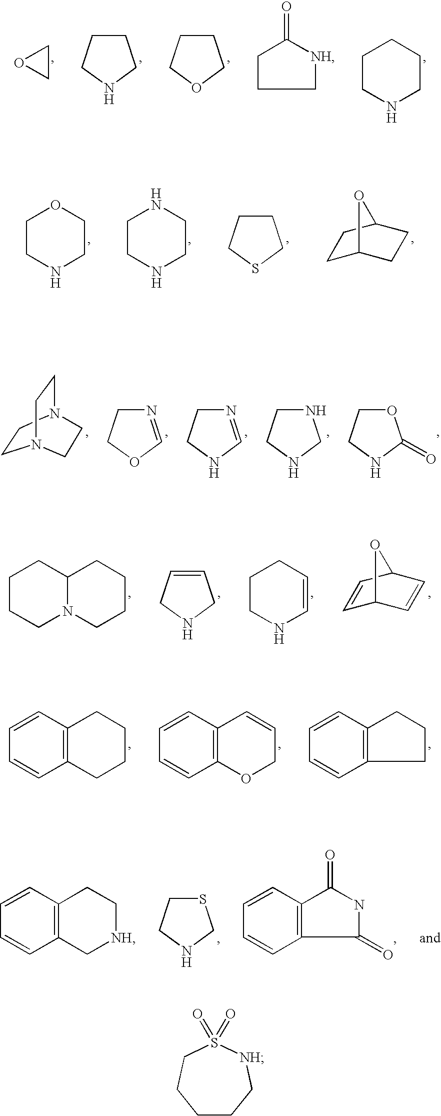

unless otherwise indicated, the foregoing (C₂-C₁₀)heterocyclyl can be C-attached or N-attached where such is possible. For instance, piperidyl can be piperid-1-yl (N-attached) or piperid-2-yl (C-attached).

Unless otherwise indicated, the term “(C₆-C₁₀)aryl” refers to an aromatic, monovalent, monocyclic or fused or unfused bicyclic or tricyclic functional group referred to herein containing a total of from 6 to 10 ring carbon atoms. Illustrative examples of (C₆-C₁₀)aryl are derived from, but not limited to, the following

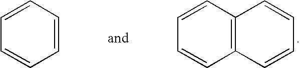

Unless otherwise indicated, the term “(C₁-C₁₀)heteroaryl” refers to an aromatic, monovalent monocyclic, fused or unfused bicyclic or tricyclic functional group referred to herein containing a total of from 1 to 10 ring carbon atoms and 1 to 5 ring heteroatoms selected from nitrogen, oxygen and sulfur. Illustrative examples of (C₁-C₁₀)heteroaryl are derived from, but not limited to, the following:

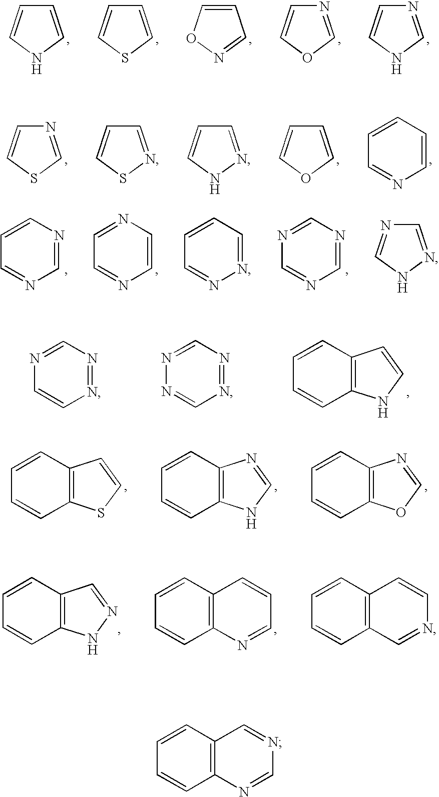

unless otherwise indicated, the foregoing (C₁-C₁₀)heteroaryl can be C-attached or N-attached where such is possible. For instance, pyridyl can be pyrid-1-yl (N-attached) or pyrid-3-yl (C-attached).

The term “a pharmaceutically acceptable salt” refers to a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

The term “treating” or “treated”, as used herein, refers to reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

The term “modulate” or “modulating”, as used herein, refers to the ability of a modulator for a member of the steroid/thyroid superfamily to either directly (by binding to the receptor as a ligand) or indirectly (as a precursor for a ligand or an inducer which promotes production of ligand from a precursor) induce expression of gene(s) maintained under hormone expression control, or to repress expression of gene(s) maintained under such control.

The term “obesity” or “obese”, as used herein, refers generally to individuals who are at least about 20-30% over the average weight for his/her age, sex and height. Technically, “obese” is defined, for males, as individuals whose body mass index is greater than 27.8 kg/m, and for females, as individuals whose body mass index is greater than 27.3 kg/m². Those of skill in the art readily recognize that the invention method is not limited to those who fall within the above criteria. Indeed, the method of the invention can also be advantageously practiced by individuals who fall outside of these traditional criteria, for example, by those who may be prone to obesity.

The term “Inflammatory disorders”, as used herein, refers to disorders such as rheumatoid arthritis, ankylosing spondylitis, psoriatic arthritis, psoriasis, chondrocalcinosis, gout, inflammatory bowel disease, ulcerative colitis, Crohn's disease, fibromyalgia, and cachexia.

The phrase “therapeutically effective amount”, as used herein, refers to that amount of drug or pharmaceutical agent that will elicit the biological or medical response of a tissue, system, animal, or human that is being sought by a researcher, veterinarian, medical doctor or other.

The phrase “amount . . . effective to lower blood glucose levels”, as used herein, refers to levels of compound sufficient to provide circulating concentrations high enough to accomplish the desired effect. Such a concentration typically falls in the range of about 10 nM up to 2 μM; with concentrations in the range of about 100 nM up to 500 nM being preferred. As noted previously, since the activity of different compounds which fall within the definition of Formula (I) as set forth above may vary considerably, and since individual subjects may present a wide variation in severity of symptoms, it is up to the practitioner to determine a subject's response to treatment and vary the dosages accordingly.

The phrase “insulin resistance”, as used herein, refers to the reduced sensitivity to the actions of insulin in the whole body or individual tissues, such as skeletal muscle tissue, myocardial tissue, fat tissue or liver tissue. Insulin resistance occurs in many individuals with or without diabetes mellitus.

The phrase “insulin resistance syndrome”, as used herein, refers to the cluster of manifestations that include insulin resistance, hyperinsulinemia, non insulin dependent diabetes mellitus (NIDDM), arterial hypertension, central (visceral) obesity, and dyslipidemia.

Other metabolic disorders associated with impaired glucose utilization and insulin resistance include major late-stage complications of NIDDM, such as diabetic angiopathy, atherosclerosis, diabetic nephropathy, diabetic neuropathy, and diabetic ocular complications such as retinopathy, cataract formation and glaucoma, and many other conditions linked to NIDDM, including dyslipidemia glucocorticoid induced insulin resistance, dyslipidemia, polycysitic ovarian syndrome, obesity, hyperglycemia, hyperlipidemia, hypercholesteremia, hypertriglyceridemia, hyperinsulinemia, and hypertension. Brief definitions of these conditions are available in any medical dictionary, for instance, Stedman's Medical Dictionary (Xth Ed.).

The phrase “processes mediated by PPAR-γ”, as used herein, refers to biological, physiological, endocrinological, and other bodily processes which are mediated by receptor or receptor combinations which are responsive to the PPAR agonists described herein (e.g., diabetes, hyperlipidemia, obesity, impaired glucose tolerance, hypertension, fatty liver, diabetic complications (e.g. retinopathy, nephropathy, neurosis, cataracts and coronary artery diseases and the like), arteriosclerosis, pregnancy diabetes, polycystic ovary syndrome, cardiovascular diseases (e.g. ischemic heart disease and the like), cell injury (e.g. brain injury induced by strokes and the like) induced by atherosclerosis or ischemic heart disease, gout, inflammatory diseases (e.g. arthrosteitis, pain, pyrexia, rheumatoid arthritis, inflammatory enteritis, acne, sunburn, psoriasis, eczema, allergosis, asthma, GI ulcer, cachexia, autoimmune diseases, pancreatitis and the like), cancer, osteoporosis and cataracts. Modulation of such processes can be accomplished in vitro or in vivo. In vivo modulation can be carried out in a wide range of subjects, such as, for example, humans, rodents, sheep, pigs, cows, and the like.

The PPAR agonists of the present invention may be administered in combination with other agents such as α-glucosidase inhibitors, aldose reductase inhibitors, biguanide preparations, statin base compounds, squalene synthesis inhibitors, fibrate base compounds, LDL catabolism promoters and angiotensin-converting enzyme inhibitors.

In the above description, an α-glucosidase inhibitor is a medicament having action in inhibiting a digestive enzyme such as amylase, maltase, α-dextrinase or sucrase, thereby retarding the digestion of starch or sucrose. Examples of α-glucosidase inhibitors include acarbose, N-(1,3-dihydroxy-2-propyl)variolamine (common name: voglibose) and miglitol.

In the above description, an aldose reductase inhibitor is a medicament which inhibits a rate-limiting enzyme of the first step of the polyol pathway, thereby inhibiting diabetic complications. Examples include tolrestat, epalrestat, 2,7-difluoro-spiro(9H-fluoren-9,4′-imidazolidine)-2′,5′-dione (common name: imirestat), 3-[(4-bromo-2-fluorophenyl)methyl]-7-chloro-3,4-dihydro-2,4-dioxo-1(2H)-quinozolineacetic acid (common name: zenarestat), 6-fluoro-2,3-dihydro-2,5′-dioxo-spiro[4H-1-benzopyran-4,4′-imidazolidine]-2-carboxamide (SNK-860), zopolrestat, sorbinil and 1-[(3-bromo-2-benzofuranyl)sulfonyl]-2,4-imidazolidinedione (M-16209).

In the above description, a biguanide preparation is a medicament having effects in anaerobic glycolysis promotion, insulin action reinforcement at the periphery, intestinal glucose absorption inhibition, hepatic gluconeogenesis inhibition and fatty-acid oxidation inhibition and examples include phenformin, mefformin and buformin.

In the above description, a statin base compound is a medicament which inhibits hydroxymethylglutaryl CoA (HMG-CoA) reductase, thereby lowering the blood cholesterol level and examples include pravastatin and the sodium salt thereof, simvastatin, lovastatin, atorvastatin and fluvastatin.

In the above description, a squalene synthesis inhibitor is a medicament for inhibiting squalene synthesis, thereby lowering the blood cholesterol level and examples include monopotassium (S)-α-[bis(2,2-dimethyl-1-oxopropoxy)methoxy]phosphinyl-3-phenoxybenzene-butanesulfonate (BMS-188494).

In the above description, a fibrate base compound is a medicament for inhibiting synthesis and secretion of triglycerides in the liver and activating lipoprotein lipase, thereby lowering the triglyceride level in the blood. Examples include bezafibrate, beclobrate, binifibrate, ciprofibrate, clinofibrate, clofibrate, clofibric acid, ethofibrate, fenofibrate, gemfibrozil, nicofibrate, pirifibrate, ronifibrate, simfibrate and theofibrate.

In the above description, a LDL catabolism promoter is a medicament for increasing LDL (low-density lipoprotein) receptors, thereby lowering the blood cholesterol level and examples include compounds described in Japanese Patent Application Kokai Hei 7-316144 or salts thereof, more specifically, N-[2-[4-bis(4-fluorophenyl)methyl-1-piperazinyl]ethyl]-7,7-diphenyl-2,4,6-heptatrienoic amide.

The above-described statin base compounds, squalene synthesis inhibitors, fibrate base compounds and LDL catabolism promoters can be replaced with another chemical effective for lowering the blood cholesterol or triglyceride level. Examples of such a medicament include nicotinic acid derivative preparations such as nicomol and niceritrol; antioxidants such as probucol; and ion exchange resin preparations such as cholestyramine.

In the above description, an angiotensin-converting enzyme inhibitor is a medicament for inhibiting angiotensin-converting enzyme, thereby lowering the blood pressure and at the same time, partially lowering the blood sugar level of a patient suffering from diabetes. Examples include captopril, enalapril, alacepril, delapril, ramipril, lisinopril, imidapril, benazepril, ceronaprill cilazapril, enalaprilat, fosinopril, moveltipril, perindopril, quinapril, spirapril, temocapril and trandolapril.

The phrase “in combination with”, as used herein, means that the fused heteroaryl compound of Formula (I) may be administered shortly before, shortly after, concurrently, or any combination of before, after, or concurrently, with such other agents as described in the previous paragraphs. Thus, the fused heteroaryl compound of Formula (I) and the other agents may be administered simultaneously as either as a single composition or as two separate compositions or sequentially as two separate compositions.

Other aspects, advantages, and preferred features of the invention will become apparent from the detailed description below.

### DETAILED DESCRIPTION AND PREFERRED EMBODIMENTS OF THE INVENTION

Compounds of Formula (I) may be prepared by the application or adaptation of known methods, by which is meant methods used heretofore or described in the literature. General methods for preparing compounds according to the invention may also be prepared as described in the Reaction Schemes that follow. Unless otherwise indicated each R¹, Ar, A, Y, HET, Q, and T in the reaction Schemes and the discussion that follows are defined as above.

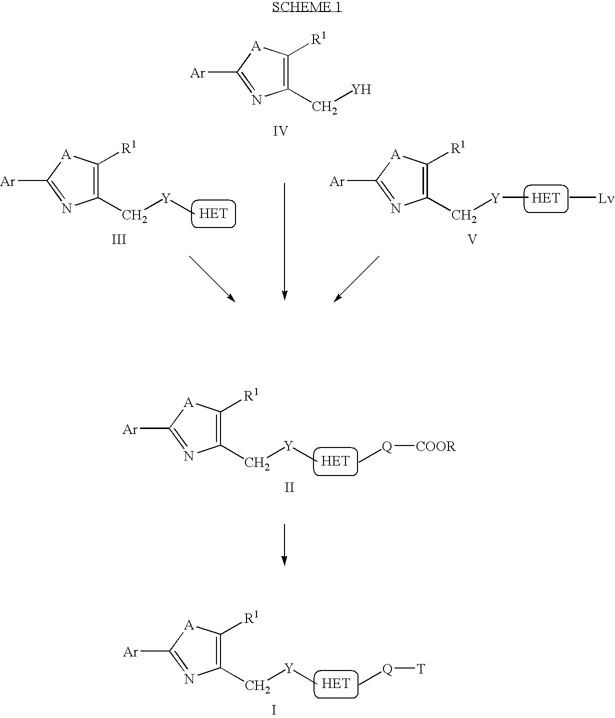

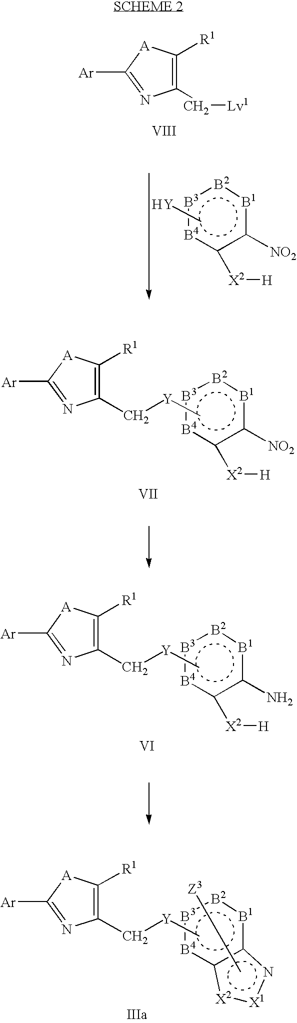

Scheme 1 refers to the preparation of compounds of the formula I. Referring to Scheme 1, a compound of formula I can be prepared by reacting a compound of the formula II, wherein the group COOR is an ester group, such as methyl ester, benzyl ester or ethyl ester, with an ester hydrolyzing agent in a solvent. Suitable ester hydrolyzing agents include bases, such as lithium hydroxide. There is no particular limitation on the nature of the solvent to be used in the above reaction provided that it can be used in ordinary hydrolysis. Examples include tetrahydrofuran. The aforesaid reaction can be generally carried out by a method known in the field of organic synthetic chemistry, for example, T. W. Green (Protective Groups in Organic Synthesis), John Wiley & Sons or J. F. W. McOmie, (Protective Groups in Organic Chemistry), Plenum Press.

Compounds of formula II can be prepared by reacting a compound of the formula III with a compound of the formula:

H—Q—COOR;

wherein COOR is as described in the previous paragraph, in a solvent. Suitable compounds of the formula H—Q—COOR include methyl acrylate and ethyl acrylate. Suitable solvents include chloroform, dioxane, tetrahydrofuran, dimethylformamide, or methylene chloride; preferably tetrahydrofuran. The aforesaid reaction can be conducted at a temperature of about 0° C. to about 25° C., preferably about 25° C. The aforesaid reaction can be conducted for a time period of about 5 minutes to about 24 hours, preferably about 5 hours.

Alternatively, compounds of formula II can be prepared reacting a compound of formula IV, with a compound of formula:

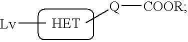

wherein COOR is as described in the previous paragraph in the presence of a suitable base and a catalyst in a non-polar solvent, such as benzene or toluene. Suitable bases include alkoxide bases (such as sodium methoxide, sodium ethoxide, or potassium tert-butoxide); hydride bases (such as sodium hydride); or carbonate bases (such as potassium carbonate or cesium carbonate); preferably potassium carbonate. Suitable catalysts include palladium acetate. The aforesaid reaction can be conducted at a temperature of about 50° C. to about 100° C., preferably about 80° C. The aforesaid reaction can be conducted for a time period of about 0.5 hour to about 72 hours, preferably about 18 hours.

Yet alternatively, compounds of formula II can be prepared reacting a compound of formula V, with a compound of formula:

H—Q—COOR;

wherein COOR is as described in the previous paragraph, in the presence of a suitable base and a catalyst in a non-polar solvent, such as benzene or toluene. Suitable compounds of the formula H—Q—COOR include benzyl acrylate and methyl acrylate. Suitable bases include amines such as triethyl amine. Suitable catalysts include palladium acetate. The aforesaid reaction can be conducted at a temperature of about 50° C. to about 100° C., preferably about 90° C. The aforesaid reaction can be conducted for a time period of about 0.5 hour to about 72 hours, preferably about 4 hours.

Certain fused heteroaryls can be prepared by ring closure reactions. Scheme 2 refers to the preparation of compounds of the formula IIIa, which is a compound of the formula III, wherein the group HET is of the formula (I). Referring to Scheme 2, a compound of formula IIIa can be prepared by reacting a compound of the formula VI with an acetate salt, such as potassium acetate, and acetic anhydride, followed by a nitrosating agent such as isoamyl nitrite, in a non-polar solvent, such as benzene.

Compounds of formula VI can be prepared by reacting a compound of the formula VII with a hydrogenating agent, such as 10% palladium on carbon, in a polar solvent such as methanol and ethyl acetate. The aforesaid reaction can be conducted at a temperature of about 0° C. to about 25° C. The aforesaid reaction can be conducted for a time period of about 5 minutes to about 24 hours, preferably about 4 hours.

Compounds of formula VII can be prepared reacting a compound of formula VIII, wherein Lv¹ is a leaving group, such as halo, preferably bromo or chloro, or p-TsO—, with a compound of formula:

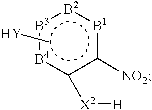

such as 3-methyl-2-nitrophenol, in a polar solvent. Suitable polar solvents include acetonitrile, tetrahydrofuran, dimethylformamide, dimethyl sulfoxide, or alcohols (such as ethanol), preferably acetonitrile. The aforesaid reaction can be conducted at a temperature of about 60° C. to about 100° C., preferably about 70° C. The aforesaid reaction can be conducted for a time period of about 3 hour to about 72 hours, preferably about 24 hours.

The invention will now be described in greater detail by reference to the following non-limiting examples.

### EXAMPLE 1

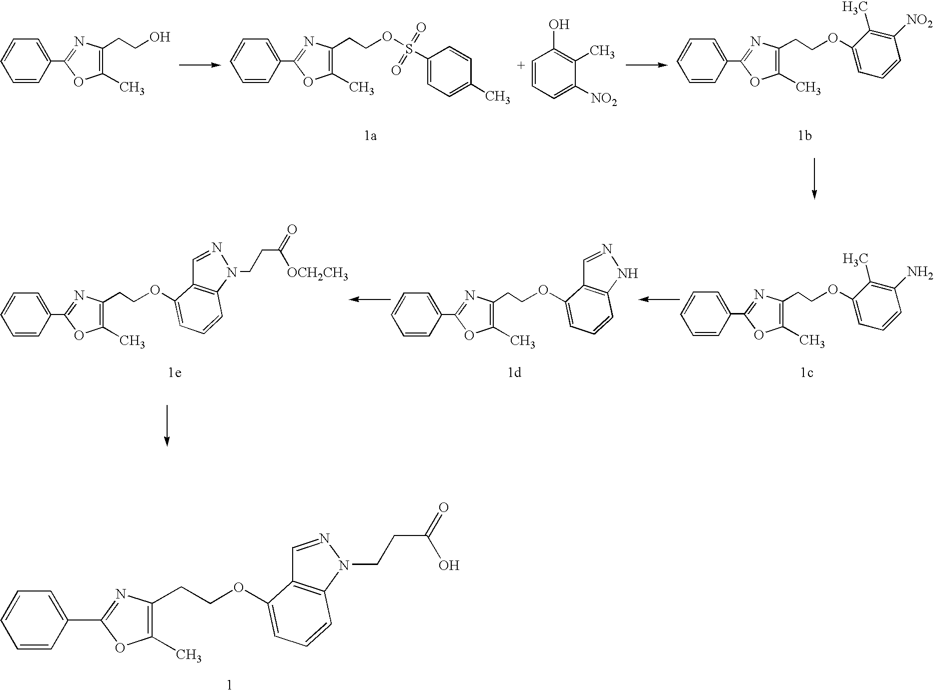

### 3-{4-[2-(5-methyl-2-phenyl-1,3-oxazol-4-yl)ethoxy]-1H-indazol-1-yl}propanoic acid

A solution of ester 1e (4.13 g, 9.83 mmol) in THF (200 mL) was treated with 30 mL of 1N LiOH at room temperature. After 5 hour stirring, the THF was removed under vacuum, and the resulting slurry was poured into an excess of 0.5M aqueous NaHSO₄, extracted with CHCl₃. The organic layer was concentrated to give the title compound as a white powder (3.93 g, XX%). ¹H NMR (DMSO-d₆) δ: 12.11 (1H, br s), 7.78 (1H, s), 7.75 (2H, dd, J=7.3 and 2.3 Hz), 7.35-7.30 (3H, m), 7.10 (1H, t, J=7.3 Hz), 7.05 (1H, d, J=8.6 Hz), 6.43 (1H, d, J=7.3 Hz), 4.37 (2H, t, J=6.6 Hz), 4.19 (2H, t, J=6.3 Hz), 2.85 (2H, t, J=6.6 Hz), 2.65 (2H, t, J=6.6 Hz), 2.23 (3H, s). LRMS (m/z) 392 (M+H)⁺. Anal. Calcd. For C₂₂H₂₁N₃O₄: C, 67.51; H, 5.41; N, 10.74. Found: C, 67.47; H, 5.49; N, 10.55.

Preparation of compound 1e: ethyl 3-{4-[2-(5-methyl-2-phenyl-1,3-oxazol-4-yl)ethoxy]-1H-indazol-1-yl}propanoate

To a solution of indazole 1d (4.47 g, 14.0 mmol) and ethyl acrylate (1.8 mL, 17 mmol)) in DMF (200 mL) was added Cs₂CO₂ (5.47 g, 16.8 mmol) and the reaction mixture was warmed to 60° C. After 1 hour stirring, the reaction was cooled to room temperature, poured into ethyl acetate and washed with H₂O (×2). The organic layer was concentrated and purified on silica gel eluting with a linear gradient elution of 0% to 5% acetone in CH₂Cl₂ to give the title compound as a white solid (4.41 g, 75%). ¹H NMR (CDCl₃) δ: 8.03 (1H, s), 7.98 (2H, dd, J=7.8 and 1.8Hz), 7.45-7.39 (3H, m), 7.26 (1H, t, J=8.1 Hz), 7.03 (1H, d, J=8.3 Hz), 6.48 (1H, d, J=7.8 Hz), 4.62 (2H, t, J=7.1 Hz), 4.40 (2H, t, J=6.6 Hz), 4.09 (2H, q, J=7.3 Hz), 3.08 (2H, t, J=6.6 Hz), 2.94 (2H, t, J=6.8 Hz), 2.42 (3H, s), 1.18 (3H, t, J=7.1 Hz). LRMS (m/z) 420 (M+H)⁺. Anal. Calcd. For C₂₄H₂₅N₃O₄: C, 68.72; H, 6.01; N, 10.02. Found: C, 68.55; H, 5.97; N, 9.98.

Preparation of compound 1d: 4-[2-(5-methyl-2-phenyl-1,3-oxazol-4-yl)ethoxy]-1H-indazole

A solution of aniline 1c (5.65 g, 18.3 mmol) in benzene (400 mL) at room temperature was treated with potassium acetate (1.98 g, 20.2 mmol) and acetic anhydride (6.9 mL, 73.1 mmol). After 1 hour stirring, a white precipitate formed, and the slurry was treated with isoamylnitrite (4.93 mL, 36.7 mmol) and brought to reflux. After 20 hours, the solvent was removed under vacuum and the residue dissolved in methanol (500 mL) and treated with K₂CO₃ (14.4 g, 146 mmol). After 4 hour stirring at room temperature, the methanol was removed under vacuum and the resulting residue was partitioned between CHCl₃ and H₂O. The aqueous layer was further extracted with CHCl₃ and the organic layers were combined, concentrated and purified on silica gel eluting with a linear gradient elution of 0% to 30% acetone in CH₂Cl₂ to give the title compound as a pale tan solid (4.52 g, 77%). ¹H NMR (DMSO-d₆) δ: 12.92 (1H, br s), 7.85 (1H, s), 7.81 (2H, dd, J=7.8 and 1.8 Hz), 7.42-7.35 (3H, m), 7.12 (1H, t, J=8.1 Hz), 6.96 (1H, d, J=8.3 Hz), 6.46 (1H, d, J=7.6Hz), 4.25 (2H, t, J=6.3 Hz), 2.92 (2H, t, J=6.3 Hz), 2.30 (3H, s). LRMS (m/z) 320 (M+H)⁺. Anal. Calcd. For C₁₉H₁₇N₃O₂.0.20H₂O: C, 70.66; H, 5.43; N, 13.01. Found: C, 70.69; H, 5.25; N, 12.77.

Preparation of compound 1c: 2-methyl-3-[2-(5-methyl-2-phenyl-1,3-oxazol-4-yl)ethoxy]aniline

A solution of compound 1b (6.37 g, 18.8 mmol) in 200 mL of 1:1 methanol/ethyl acetate was treated with 10% Pd/C (0.60 g) and hydrogenated under 40 psi of H₂. After 1 hour, the reaction mixture was filtered through Celite® and concentrated to give a light tan solid, which was used without further purification (5.68 g, 98%). ¹H NMR (DMSO-d₆) δ: 7.81 (2H, dd, J=7.6 and 2.0 Hz), 7.28-7.21 (3H, m), 6.55 (1H, t, J=8.1 Hz), 6.00 (1H, d, J=7.8 Hz), 5.96 (1H, d, J=7.8 Hz), 4.52 (2H, s), 3.86 (2H, t, J=6.3), 2.66 (2H, t, J=6.3 Hz), 2.12 (3H, s), 1.60 (3H, s). LRMS (m/z) 309 (M+H)⁺. Anal. Calcd. For C₁₉H₂₀N₂O₂: C, 74.00; H, 6.54; N, 9.08. Found: C, 73.80; H, 6.55; N, 9.08.

Preparation of compound 1b: 5-methyl-4-[2-(2-methyl-3-nitrophenoxy)ethyl]-2-phenyl-1,3-oxazole

To a solution of tosylate 1a (5.21 g, 14.6 mmol) in acetonitrile (100 mL) at room temperature was added 3-methyl-2-nitrophenol (3.35g, 21.9 mmol) and then Cs₂CO₃ (7.13 g, 21.9 mmol). After 3 hour stirring at 70° C. and an additional 16 hour at 60° C., the acetonitrile was removed under vacuum and the resulting residue was partitioned between ethyl acetate and H₂O. The organic layer was washed with 1N NaOH (×4), concentrated, and the crude residue was purified on silica gel eluting with a linear gradient elution of 0% to 30% ethyl acetate in hexanes to give the title compound as a pale yellow solid (3.83 g, 78%). ¹H NMR (CDCl₃) δ: 7.99-7.96 (2H, m), 7.46-7.36 (4H, m), 7.23 (1H, t, J=7.8 Hz), 7.06 (1H, d, J=8.1 Hz), 4.30 (2H, d, J=6.3 Hz), 3.03 (2H, t, J=6.6), 2.39 (3H, s), 2.33 (3H, s). HRMS calculated for C₁₉H₁₉N₂O₄ 339.1340 (M+H)⁺, found 339.1352. Anal. Calcd. For C₁₉H₁₈N₂O): C, 67.45; H, 5.36; N, 8.28. Found: C, 67.20; H, 5.36; N, 8.27.

Preparation of compound 1a: 2-(5-methyl-2-phenyl-1,3-oxazol-4-yl)ethyl 4-methylbenzenesulfonate

To a solution of 2-(5-methyl-2-phenyl-1,3-oxazol-4-yl)ethanol (5.0 g, 25 mmol) in pyridine (100 mL) at 0° C. was added p-toluenesulfonic anhydride in one portion. After 1 hour at room temperature, the reaction was quenched with H₂O (10 mL) for 15 minutes and then concentrated under vacuum. The crude residue was purified on silica gel eluting with a linear gradient elution of 0% to 30% ethyl acetate in hexanes to give the title compound as a white solid (7.58 g, 86%). ¹H NMR (CDCl₃) δ: 7.88-7.85 (2H, m), 7.66 (2H, d, J=8.3 Hz), 7.43-7.40 (3H, m), 7.18 (2H, d, J=7.8 Hz), 4.31 (1H, t, J=6.3 Hz), 2.82 (2H, t, J=6.3 Hz), 2.30 (3H, s), 2.20 (3H, s). HRMS calculated for C₁₉H₂₀N₂O₄S₁ 358.1108 (M+H)⁺, found 358.1108.

### EXAMPLES 2A AND 2B

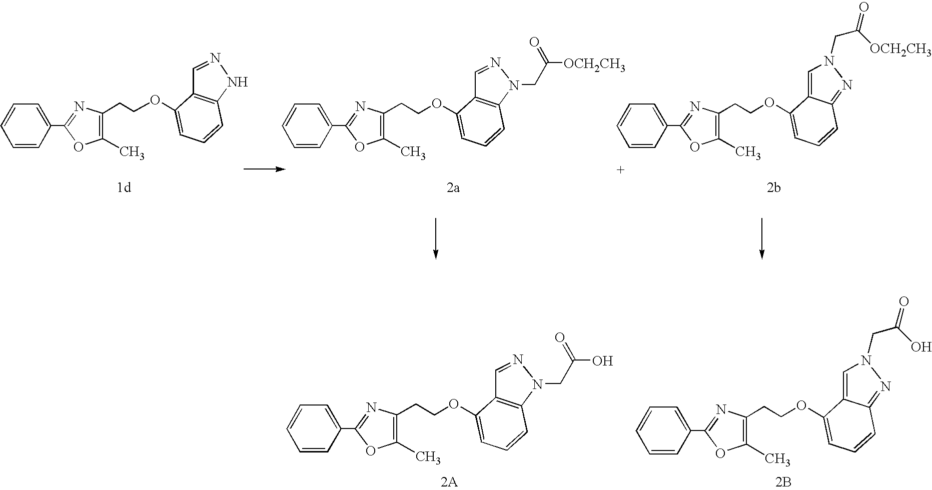

### EXAMPLE 2A

### {4-[2-(5-methyl-2-phenyl-1,3-oxazol-4yl)ethoxy]-1H-indazol-1-yl}acetic acid

Compound 2a was hydrolyzed as described in Example 1 to give the title compound 2A as a white powder (116 mg, 83%). ¹H NMR (DMSO-d₆) δ. 7.78 (1H, s), 7.74-7.72 (2H, m), 7.33-7.28 (3H, m), 7.08 (1H, t, J=7.8 Hz), 6.97 (1H, d, J=8.3 Hz), 6.44 (1H, d, J=7.6 Hz), 5.02 (2H, s), 4.19 (2H, t, J=6.6 Hz), 2.84 (2H, t, J=6.3 Hz), 2.22 (3H, s). LRMS (m/z) 378 (M+H)⁺.

### EXAMPLE 2B

### {4-[2-(5-methyl-2-phenyl-1,3-oxazol-4-yl)ethoxy]-2H-indazol-2-yl}acetic acid

Compound 2b was hydrolyzed as described in Example 1 to give the title compound 2B as a white powder (59 mg, 100%). ¹H NMR (DMSO-d₆) δ 8.26 (1H, s), 7.89-7.86 (2H, m), 7.48-7.86 (3H, m), 7.09-7.07 (2H, m), 6.40 (1H, dd, J=5.56 and 2.5 Hz), 5.02 (2H, s), 4.29 (2H, t, J=6.3 Hz), 2.98 (2H, t, J=6.1 Hz), 2.38 (3H, s). LRMS (m/z) 378 (M+H)⁺.

Preparation of compound 2a: ethyl {4-[2-(5-methyl-2-phenyl-1,3-oxazol-4-yl)ethoxy]-1H-indazol-1-yl}acetate and 2b: ethyl {4-[2-(5-methyl-2-phenyl-1,3-oxazol-4-yl)ethoxy]-2H-indazol-2-yl}acetate

To a solution of 4-[2-(5-methyl-2-phenyl-1,3-oxazol-4-yl)ethoxy]-1H-indazole (0.228 g, 0.714 mmol) and ethyl bromoacetate (0.1 mL, 0.9 mmol) in DMF (15 mL) at room temperature was added Cs₂CO₃. After 16 hour stirring, the reaction mixture was poured into ethyl acetate, washed with H₂O (×2), concentrated and purified on silica gel eluting with a gradient elution of 5% to 40% ethyl acetate in hexanes to give the title compounds iia (0.149 g, 52%) and iib (0.052 g, 18%) as white solids.

2a: LRMS (m/z) 378 (M+H)⁺.

2b: LRMS (m/z) 378 (M+H)⁺.

### EXAMPLE 3

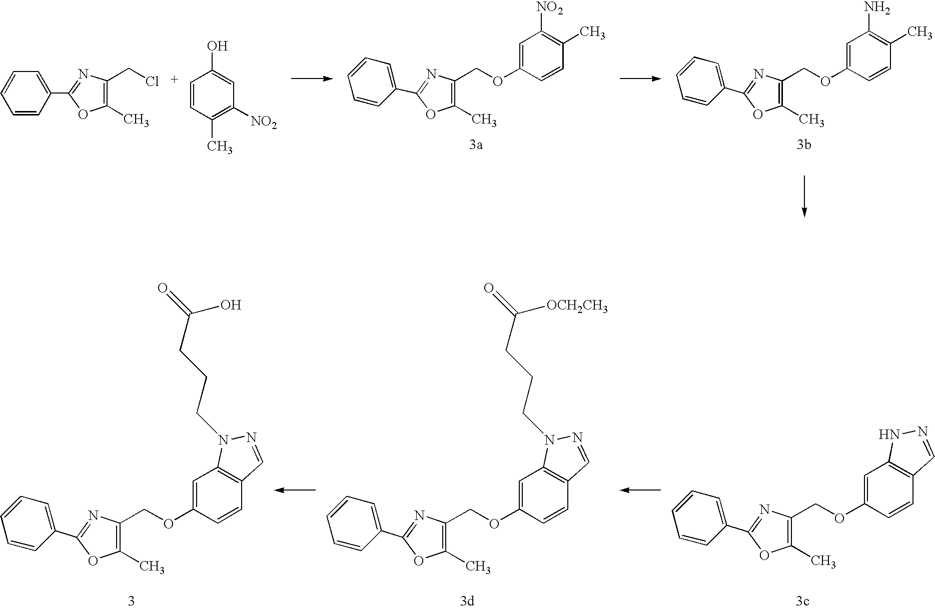

### EXAMPLE 3

### 4-{6-[(5-methyl-2-phenyl-1,3-oxazol-4-yl)methoxy]-1H-indazol-1-yl}butanoic acid

Title compound example 3 was prepared as described in Example 1, using compound 3d as the starting material to give 51.7 mg of product as a white solid. ¹H NMR (CDCl₃) δ: 8.01-7.98 (2H, m), 7.88 (1H, s), 7.57-7.55 (1H, m), 7.45-7.41 (3H, m), 7.00-6.99 (1H, m), 6.88-6.85 (1H, m), 5.08 (2H, s), 4.41 (2H, t, J=6.8 Hz), 2.44 (3H, s), 2.31 (2H, t, J=6.8 Hz), 2.23-2.26 (2H, m). LRMS (m/z) 392 (M+H)⁺. HRMS (m/z) Calcd. For C₂₂H₂₁N₃O₄ (M+H)⁺: 392.1605. Found: 392.1598.

Preparation of compound 3d: ethyl 4-{6-[(5-methyl-2-phenyl-1,3-oxazol-4-yl)methoxy]-1H-indazol-1-yl}butanoate

Title compound 3d was prepared as described in Example 2, using compound 3c as the starting material to give the product as a white solid (56.6 mg, 21%). ¹H NMR (MeOD₄) δ: 8.00-7.91 (3H, m), 7.65-7.62 (1H, m), 7.50-7.48 (3H, m), 7.18 (1H, s), 6.90-6.87 (1H, m), 5.11 (2H, s), 4.46-4.40 (2H, m), 4.07-4.00 (2H, m), 2.29-2.22 (2H, m), 2.20-2.16 (2H, m), 1.21-1.15 (3H, m). LRMS (m/z) 420 (M+H)⁺.

Preparation of compound 3c: 6-[(5-methyl-2-phenyl-1,3-oxazol-4-yl)methoxy]-1H-indazole

Title compound 3c was prepared as described in Example 1, using compound 3b as the starting material to give the product as an off-white solid (552 mg, 26%). ¹H NMR (MeOD₄) δ: 8.02-7.99 (2H, m), 7.94 (1H, s), 7.66-7.63 (1H, m), 7.51-7.47 (3H, m), 7.10-7.11 (1H, m), 6.89-6.86 (1H, m), 5.07 (2H, s), 2.48 (3H, s). LRMS (m/z) 306 (M+H)⁺.

Preparation of compound 3b: 2-methyl-5-[(5-methyl-2-phenyl-1,3-oxazol-4-yl)methoxy]aniline

Title compound 3b was prepared as described in Example 1, using compound 3a as the starting material to give the product as a yellow oil. LRMS (m/z) 295 (M+H)⁺.

Preparation of compound 3a: 5-methyl-4[(4-methyl-3-nitrophenoxy)methyl]-2-phenyl-1,3-oxazole

To a solution of 4-(chloromethyl)-5-methyl-2-phenyl-1,3-oxazole (10.0 g, 48.15 mmol) in DMF (100 mL) was added 4-methyl-3-nitrophenol, followed by K₂CO₃ (7.98 g, 57.78 mmol) in one portion. The reaction was heated at 70° C. for 16 hours. Afterwards, the solvent was removed under vacuum and the residue was suspended in 300 mL of water. The aqueous layer was extracted with ethyl acetate (×3). The combined organic layers were dried (MgSO₄), filtered and concentrated under vacuum. The crude residue was purified on silica gel eluting with chloroform to give the title compound as a yellow solid (9.66 g, 61%). ¹H NMR (MeOD₄) δ: 8.02-7.99 (2H, m), 7.69-7.68 (1H, m), 7.52-7.50 (2H, m), 7.38-7.35 (1H, m), 7.29-7.25 (1H, m), 5.09 (2H, s), 3.36 (3H, s), 2.50 (3H, s). LRMS (m/z) 325 (M+H)⁺.

The compounds below were made via the procedures outlined above for Examples 1-3.

| | | | MS Data |
|---|---|---|---|
| | | | (m/z) |
| Example # | Structure | Name | for (M + H)⁺ |
| 4 | 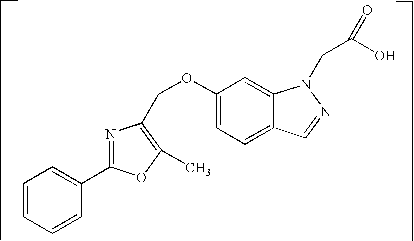 | {6-[(5-methyl-2-phenyl-1,3- oxazol-4-yl)methoxy]-1H- indazol-1-yl}acetic acid | 364 |
| 5 | 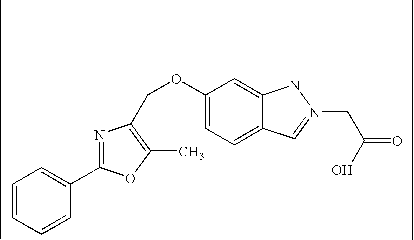 | {6-[(5-methyl-2-phenyl-1,3- oxazol-4-yl)methoxy]-2H- indazol-2-yl}acetic acid | 364 |
| 6 | 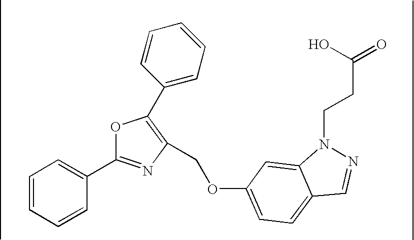 | 3-{6-[(2,5-diphenyl-1,3- oxazol-4-yl)methoxy]-1H- indazol-1-yl}propanoic acid | 440 |
| 7 | 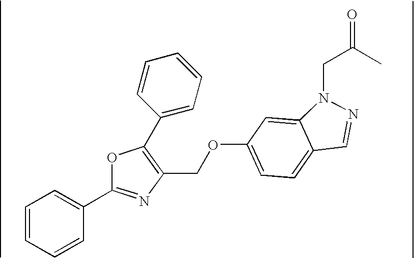 | {6-[(2,5-diphenyl-1,3- oxazol-4-yl)methoxy]-1H- indazol-1-yl}acetic acid | 426 |
| 8 | 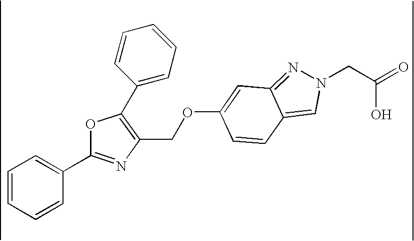 | {6-[(2,5-diphenyl-1,3- oxazol-4-yl)methoxy]-2H- indazol-2-yl}acetic acid | 426 |
| 9 | 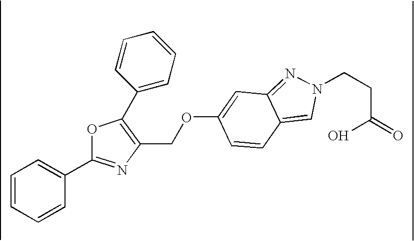 | 3-{6-[(2,5-diphenyl-1,3- oxazol-4-yl)methoxy]-2H- indazol-2-yl}propanoic acid | 440 |
| 10 | 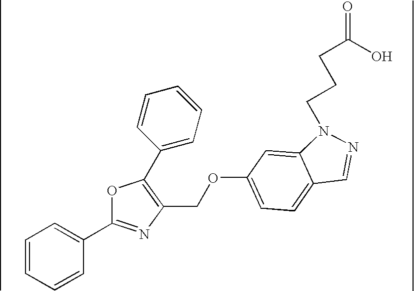 | 4-{6-[(2,5-diphenyl-1,3- oxazol-4-yl)methoxy]-1H- indazol-1-yl}butanoic acid | 454 |
| 11 | 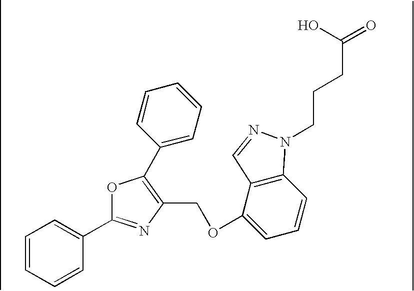 | 4-{4-[(2,5-diphenyl-1,3- oxazol-4-yl)methoxy]-1H- indazol-1-yl}butanoic acid | 454 |
| 12 | 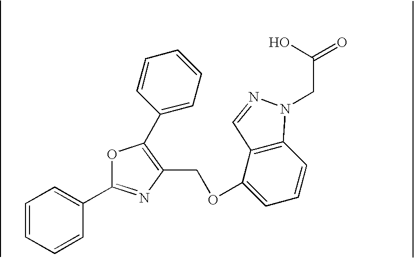 | {4-[(2,5-diphenyl-1,3- oxazol-4-yl)methoxy]-1H- indazol-1-yl)acetic acid | 426 |
| 13 | 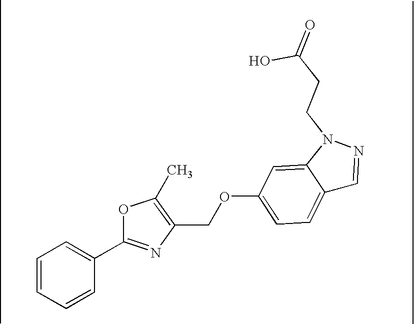 | 3-{6-[(5-methyl-2-phenyl- 1,3-oxazol-4-yl)methoxy]- 1H-indazol-1-yl}propanoic acid | 378 |
| 14 | 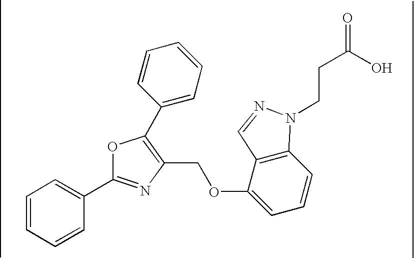 | 3-{4-[(2,5-diphenyl-1,3- oxazol-4-yl)methoxy]-1H- indazol-1-yl}propanoic acid | 441 |
| 15 | 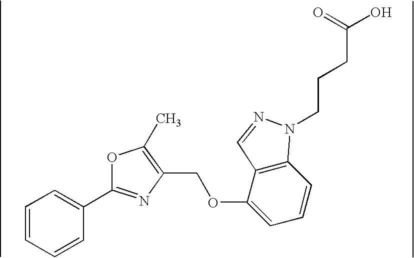 | 4-{4-[(5-methyl-2-phenyl- 1,3-oxazol-4-yl)methoxy]- 1H-indazol-1-yl}butanoic acid | 392 |
| 16 | 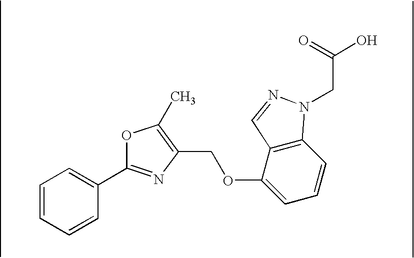 | {4-[(5-methyl-2-phenyl-1,3- oxazol-4-yl)methoxy]-1H- indazol-1-yl}acetic acid | 364 |
| 17 | 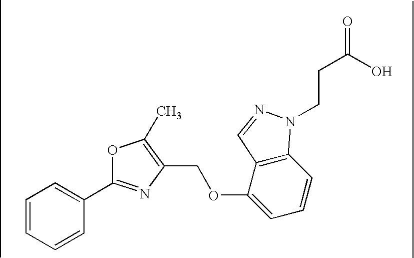 | 3-{4-[(5-methyl-2-phenyl- 1,3-oxazol-4-yl)methoxy]- 1H-indazol-1-yl}propanoic acid | 378 |
| 18 | 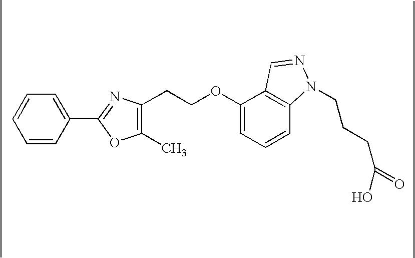 | 4-[2-(5-methyl-2-phenyl- 1,3-oxazol-4-yl)ethoxy]-1H- indazol-1-yl}butanoic acid | 406 |
| 19 | 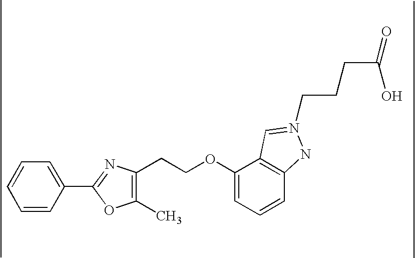 | 4-{4-[2-(5-methyl-2-phenyl- 1,3-oxazol-4-yl)ethoxy]-2H- indazol-2-yl}butanoic acid | 406 |
| 20 | 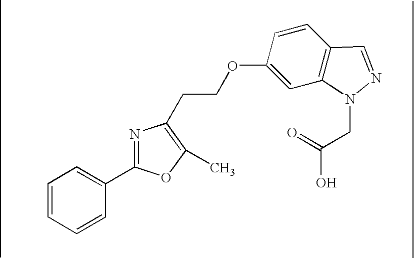 | {6-[2-(5-methyl-2-phenyl- 1,3-oxazol-4-yl)ethoxy]-1H- indazol-1-yl}acetic acid | 378 |
| 21 | 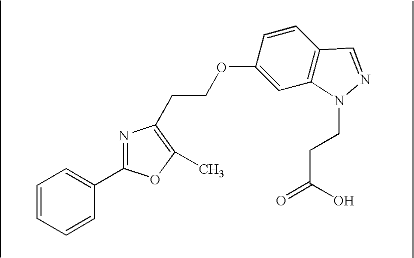 | 3-{6-[2-(5-methyl-2-phenyl- 1,3-oxazol-4-yl)ethoxy]-1H- indazol-1-yl}propanoic acid | 392 |
| 22 | 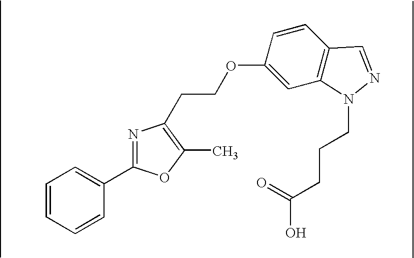 | 4-{6-[2-(5-methyl-2-phenyl- 1,3-oxazol-4-yl)ethoxy]-1H- indazol-1-yl}butanoic acid | |
| 23 | 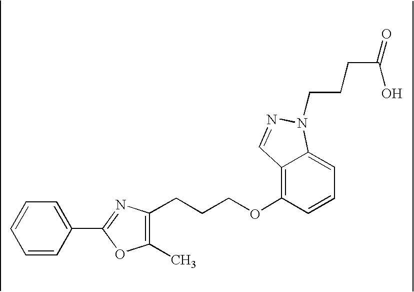 | 4-{4-[3-(5-methyl-2-phenyl- 1,3-oxazol-4-yl)propoxy]- 1H-indazol-1-yl}butanoic acid | 420 |
| 24 | 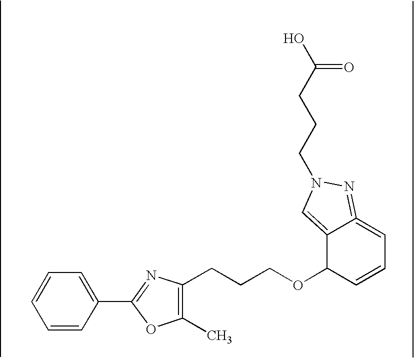 | 4-{4-[3-(5-methyl-2-phenyl- 1,3-oxazol-4-yl)propoxy]- 2H-indazol-2-yl}butanoic acid | 420 |
| 25 | 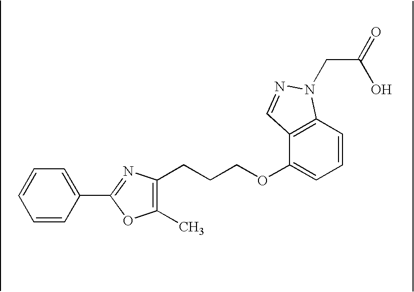 | {4-[3-(5-methyl-2-phenyl- 1,3-oxazol-4-yl)propoxy]- 1H-indazol-1-yl}acetic acid | 392 |
| 26 | 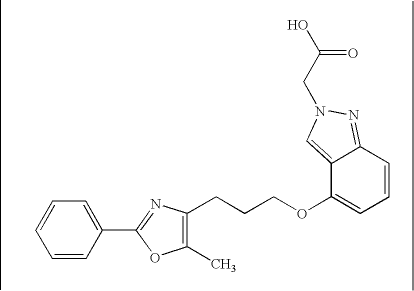 | {4-[3-(5-methyl-2-phenyl- 1,3-oxazol-4-yl)propoxy]- 2H-indazol-2-yl}acetic acid | 392 |
| 27 | 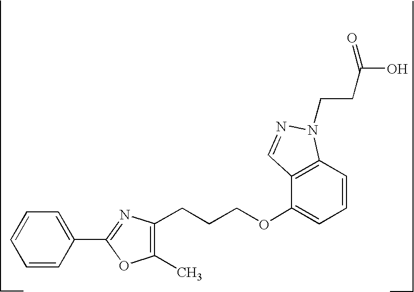 | 3-{4-[3-(5-methyl-2-phenyl- 1,3-oxazol-4-yl)propoxy]- 1H-indazol-1-yl}propanoic acid | 406 |

### EXAMPLE 28

Preparation of compound 28: 3-{4-[2-(5-methyl-2-phenyl-1,3-oxazol-4-yl)ethoxy]-1H-pyrrolo[2,3-b]pyridin-1-yl}propanoic acid

To a solution of the product of compound 28b (34 mg, 0.08 mmol) in THF (0.4 mL) and methanol (0.1 mL) was added 2M lithium hydroxide (3 equiv). The resulting mixture stirred at 23° C. for 1 hour before water (3 mL) was added. The pH was adjusted to 5 with 1N hydrochloric acid at 0° C. After stirring at 0° C. for 10 minutes, the white precipitate was collect by filtration and dried under air to give the title compound (13.3 mg, 42%). LRMS (m/z) 392 (M+H)⁺.

Preparation of compound 28b: ethyl 3-{4-[2-(5-methyl-2-phenyl-1,3-oxazol-4-yl}ethoxy]-1H-pyrrolo[2,3-b]pyridin-1-yl}propanoate

To a solution of compound 28a (51 mg, 0.2 mmol) in toluene (1 mL) were added 2-(5-methyl-2-phenyl-1,3-oxazol-4-yl)ethanol (82 mg, 2 equiv), palladium acetate (3.6 mg, 0.08 equiv), cesium carbonate (142 mg, 2 equiv) and 2-(di-tert-butylphosphino)-1,1′-binaphthyl (8 mg, 0.1 equivalent) (Strem Chemicals). The resulting mixture was heated at 80° C. for 18 hours. The reaction mixture was poured into saturated sodium bicarbonate and extracted with ethyl acetate (3×10 mL). The combined organics were washed with saturated sodium chloride, dried over sodium sulfate, filtered, concentrated and the residue purified by silica gel chromatography using 0 to 25% ethyl acetate in hexane provided the title compound (34 mg, 40%) LRMS (m/z) 420 (M+H)⁺.

Preparation of compound 28a: ethyl 3-(4-chloro-1H-pyrrolo[2,3-b]pyridin-1-yl)propanoate

To a solution of 4-chloro-7-azaindole (59 mg, 0.39 mmol) (Clark, B. A., et al, J. Chem. Soc., Perkin I, 2270 (1974)) in N,N-dimethylformamide (DMF, 3 mL) were added cesium carbonate (318 mg, 2 equiv) and ethyl 3-bromopropionate (77 mg, 1.1 equiv). The resulting solution was stirred at 23° C. for 1 hour. The reaction mixture was poured into saturated aqueous sodium bicarbonate and extracted with ethyl acetate (3×10 mL). The combined organics were washed with water (1×20 mL) and saturated sodium chloride solution (1×20 mL), dried over sodium sulfate, filtered, evaporated and purified by silica gel chromatography to give the title compound (77 mg, 79%). LRMS (m/z) 254 (M+H)⁺.

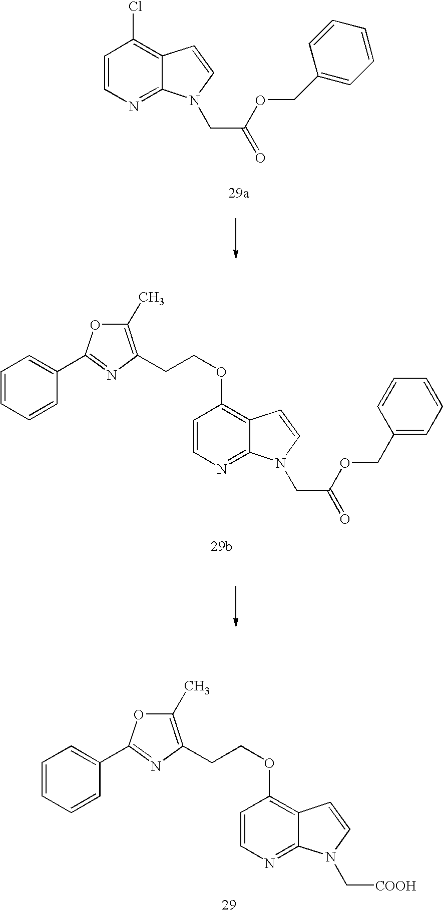

Preparation of compound 29: {4-[2-(5-methyl-2-phenyl-1,3-oxazol-4-yl)ethoxy]-1H-pyrrolo[2,3-b]pyridin-1-yl}acetic acid

To a solution of compound 29b (8 mg) in methanol (2 mL) was added 10% palladium on carbon (2 mg). The mixture stirred under hydrogen for 3 hr. Filtration through Celite® and concentration provided the title compound (6.5 mg, 100%). LRMS (m/z) 378 (M+H)⁺.

Preparation of compound 29b: benzyl {4-[2-(5-methyl-2-phenyl-1,3-oxazol-4-yl)ethoxy]-1H-pyrrolo[2,3-b]pyridin-1-yl}acetate

Following the procedures described in Preparation of compound 28b, using compound 29a in place of compound 28a, the title compound was obtained. LRMS (m/z) 467 (M+H)⁺.

Preparation of compound 29a: benzyl (4-chloro-1H-pyrrolo[2,3-b]pyridin-1-yl)acetate

Following the procedures described in Example 8, using benzyl 2-bromoacetate in place of ethyl 3-bromopropionate, the title compound 29a was prepared in 80% yield. LRMS (m/z) 301 (M+H)⁺.

### EXAMPLE 30

Preparation of compound 30: ({1-[2-(5-methyl-2-phenyl-1,3-oxazol-4-yl)ethyl]-1H-pyrrolo[2,3-b]pyridin-4-yl}oxy)acetic acid

Following the procedures described in Example 29, using compound 30b as a starting material, the title compound was obtained in 100% yield. LRMS (m/z) 378 (M+H)⁺.

Preparation of compound 30b: benzyl ({1-[2-(5-methyl-2-phenyl-1,3-oxazol-4-yl)ethyl]-1H-pyrrolo[2,3-b]pyridin-4-yl}oxy)acetate

Following the procedures described in the preparation of compound 28b, using compound 30a in place compound 28a, and benzyl 2-hydroxyacetate in place of 2-(5-methyl-2-phenyl-1,3-oxazol-4-yl)ethanol, the title compound was obtained in 20% yield. LRMS (m/z) 467 (M+H)⁺.

Preparation of compound 30a: 4-chloro-1-[2-(5-methyl-2-phenyl-1,3-oxazol-4-yl)ethyl]-1H-pyrrolo[2,3-b]pyridine

Following the procedures described in the preparation of compound 28a, using 2-(5-methyl-2-phenyl-1,3-oxazol-4-yl)-ethyl-4-methylbenzenesulfonate in place of ethyl 3-bromopropionate, the title compound was prepared in 95% yield. LRMS (m/z) 338 (M+H)⁺.

### EXAMPLE 31

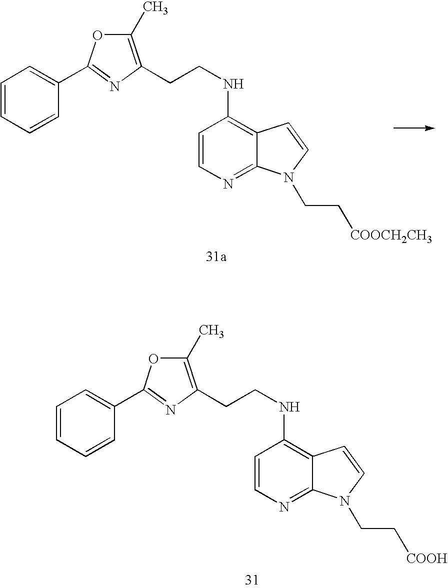

Preparation of compound 31: 3-(4-{[2-(5-methyl-2-phenyl-1,3-oxazol-4yl)ethyl]amino}-1H-pyrrolo[2,3-b]pyridin-1-yl)propanoic acid

Following the procedures described in Example 28, using the compound 31a in place of compound 28b, the title compound was obtained in 29% yield. LRMS (m/z) 391 (M+H)⁺.

Preparation of compound 31a: ethyl 3-(4-{[2-(5-methyl-2-phenyl-1,3-oxazol-4-yl)ethyl]amino}-1H-pyrrolo[2,3-b]pyridin-1-yl)propanoate

Following the procedures described in preparation of compound 28b, using 2-(5-methyl-2-phenyl-1,3-oxazol-4-yl)ethylamine (prepared from the corresponding tosylate using conventional method) in place of 2-(5-methyl-2-phenyl-1,3-oxazol-4-yl)ethanol, and 2-dicyclohexylphosphino-2′-(N,N-dimethylamino)-biphenyl in place of 2-(di-t-butylphosphino)-1,1′-binaphthyl, compound 31a was prepared in 15% yield. LRMS (m/2) 419 (M+H)⁺.

### EXAMPLE 32

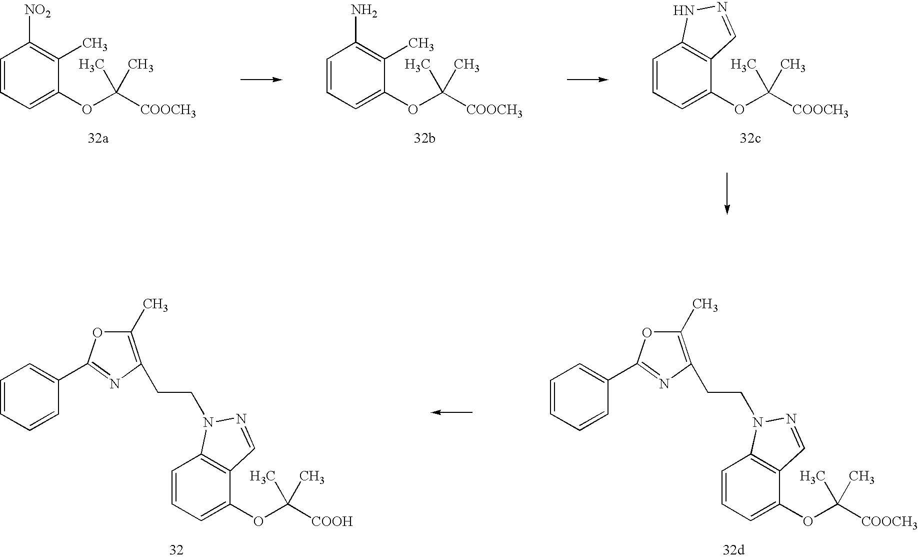

Preparation of compound 32: 2-methyl-2-({1-[2-(5-methyl-2-phenyl-1,3-oxazol-4-yl)ethyl]-1H-indazol-4yl}oxy)propanoic acid

To a solution of compound 32d (52 mg, 0.124 mmol) in methanol (1 mL) were added potassium carbonate (34 mg, 2 equiv) and water (0.5 mL). The resulting mixture heated at 80° C. for 12 hours. The mixture was diluted with water (5 mL), acidified to pH 2 with 1N hydrochloric acid and extracted with ethyl acetate (3×5 mL). The combined organics were washed with saturated sodium chloride, dried over sodium sulfate and concentrated to complete dryness to produce the title compound (50 mg, 100%). LRMS (m/z) 406 (M+H)⁺.

Preparation of compound 32d: methyl 2-methyl-2-({1-[2-(5-methyl-2-phenyl-1,3-oxazol-4-yl)ethyl]-1H-indazol-4-yl}oxy)propanoate

Following the procedures described in preparation of compound 28a, and compound 32c and 2-(5-methyl-2-phenyl-1,3-oxazol-4-yl)-ethyl-4-methylbenzenesulfonate as starting materials, the title compound was obtained in 46.6% yield. LRMS (m/z) 420 (M+H)⁺.

Preparation of compound 32c: methyl 2-(1H-indazol-4-yloxy)-2-methylpropanoate

Step 1: To a solution of the compound 32b (0.35 g, 1.57 mmol) in benzene (10 mL) were added potassium acetate (170 mg, 1.1 equiv) and acetic anhydride (0.45 mL, 3 equiv). The resulting mixture stirred at 23° C. for 30 minutes. LCMS at this point of time indicated complete acetylation. To this mixture was added isoamylnitrite (276 mg, 1.5 equiv) and the mixture was heated at 80° C. for 4 hours. The white solid was filter off and the filtrate concentrated to dryness to give the crude indazole 1-acetate which was used without further purification.

Step 2: The product of the Step 1 was dissolved in methanol (5 mL) and treated with potassium carbonate (43 mg, 0.2 equiv) at 23° C. for 12 hours. Filtration, concentration and silica gel chromatography provided the title compound 32c (288 mg, 78% over 2 steps). LRMS (m/z) 235 (M+H)⁺.

Preparation of compound 32b: methyl 2-(3-amino-2-methylphenoxy)-2-methylpropanoate

To a solution of compound 32a (0.36 g) in methanol (10 mL) and ethyl acetate (10 mL) was added 10% palladium on carbon (72 mg). The resulting mixture stirred under hydrogen for 4 hours. Filtration through Celite® and concentration provided the title compound (0.35 g, 100%). LRMS (m/z) 224 (M+H)⁺.

Preparation of compound 32a: methyl 2-methyl-2-(2-methyl-3-nitrophenoxy)propanoate

To a solution of 2-methyl-3-nitrophenol (300 mg, 2 mmol) in DMF (6 mL) were added potassium carbonate (0.55 g, 2 equiv) and methyl 2-bromo-2-methyl-proprionate (0.31 mL, 1.2 equiv). The resulting mixture was heated at 95° C. for 3 days. After cooling to 23° C., the mixture was poured into water and extracted with ethyl acetate (3×20 mL). The combined organics were washed with water and saturated sodium chloride, dried over sodium sulfate and evaporated. The residue was purified by silica gel purification using 0-15% ethyl acetate in hexane to give the title compound (367 mg, 75%). LRMS (m/z) 254 (M+H)⁺

### EXAMPLE 33

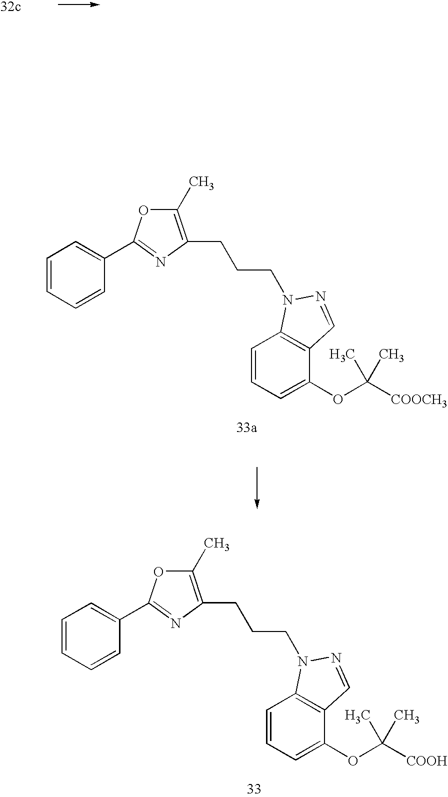

Preparation of compound 30: 2-methyl-2-({1-[3-(5-methyl-2-phenyl-1,3-oxazol-4-yl)propyl]-1H-indazol-4-yl}oxy)propanoic acid

Following the procedures described in Example 32, using compound 33a as starting material, compound 33 was obtained in 88% yield. LRMS (m/z) 420 (M+H)⁺.

Preparation of compound 33a: methyl 2-methyl-2-({1-[3-(5-methyl-2-phenyl-1,3-oxazol-4-yl)propyl]-1H-indazol-4-yl}oxy)propanoate

Following the procedures described in preparation of compound 28a, and using compound 32c and 2-(5-methyl-2-phenyl-1,3-oxazol-4-yl)-propyl-4-methylbenzenesulfonate as starting materials, the title compound 33a was obtained in 58% yield. LRMS (m/z) 434 (M+H)⁺.

### EXAMPLE 34

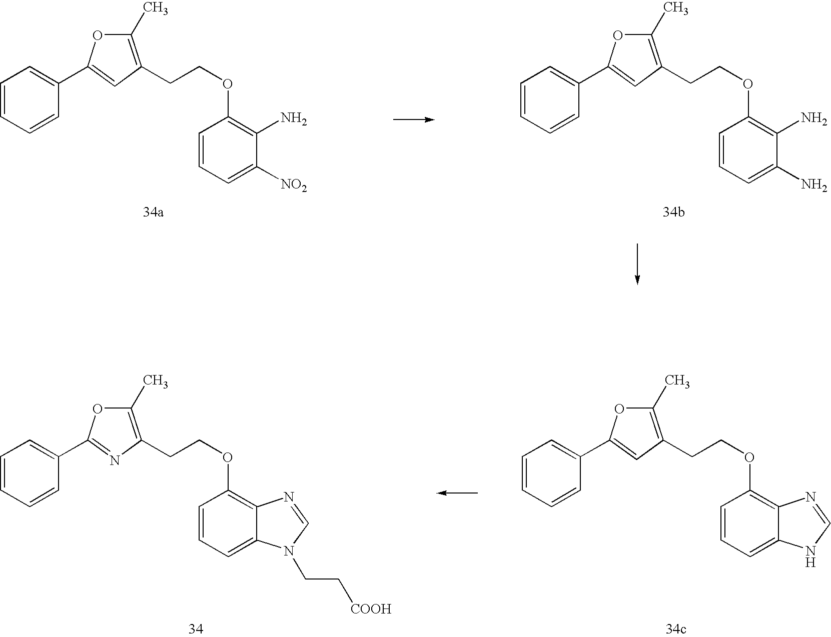

Preparation of compound 34: 3-{4-[2-(5-methyl-2-phenyl-1,3-oxazol-4-yl)ethoxy]-1H-benzimidazol-1-yl}propanoic acid

Step 1: To a solution of compound 34c and benzyl acrylate (3 equiv) in DMF was added cesium carbonate (1 equiv). The mixture was heated under microwave at 100° C. for 10 minutes.

Step 2: To the mixture of the Step 1 was added 2N lithium hydroxide (2 equivalents). After stirring at 23° C. for 1 hour, the mixture was purified by reverse phase HPLC to provide the title compound 34. LRMS (m/z) 392 (M+H)⁺.

Preparation of compound 34c: 4-[2-(2-methyl-5-phenyl-3-furyl)ethoxy]-1H-benzimidazole

Compound 34b was dissolved in formic acid and the resulting solution was heated under microwave at 100° C. for 10 minutes. After cooling, solvent was removed in vacuo to give the title compound 34c in 88% yield. LRMS (m/z) 320 (M+H)⁺.

Preparation of compound 34b: 3-[2-(2-methyl-5-phenyl-3-furyl)ethoxy]benzene-1,2-diamine

Following the procedures described in preparation of compound 32b, using compound 34a in place of compound 32a as the starting material, the title compound was prepared in quantitative yield. LRMS (m/z) 310 (M+H)⁺.

Preparation of compound 34a: 2-[2-(2-methyl-5-phenyl-3-furyl)ethoxy]-6-nitroaniline

To a solution of 2-amino-3-nitrophenol (0.4 g, 2.5 mmol) and 2-(5-methyl-2-phenyl-1,3-oxazol-4-yl)-ethanol (0.5 g, 2.5 mmol) in tetrahydrofuran (THF, 10 mL) were added triphenylphosphine (0.66 g, 2.5 mmol) and diisopropyl azodiacetate (0.66 g, 2.5 mmol) at 0° C. The resulting mixture stirred at 23° C. over night. The mixture was concentrated and the residue purified by silica gel chromatography to give the title compound (488 mg, 57%). LRMS (m/z) 340 (M+H)⁺.

### EXAMPLE 35

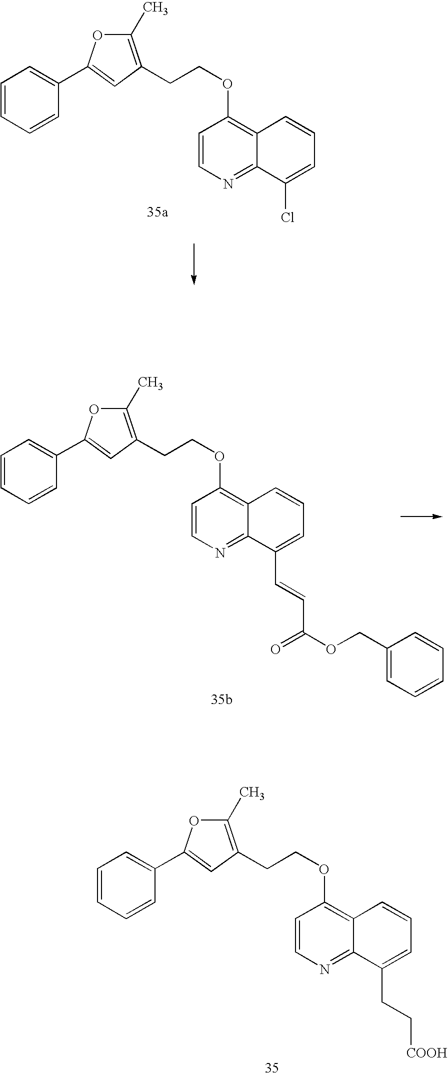

Preparation of compound 35: 3-{4-[2-(2-methyl-5-phenyl-3-furyl)ethoxy]quinolin-8-yl}propanoic acid

Following the procedures described in Example 29, using compound 35b in place of compound 29b as the starting material, the title compound 35 was obtained in quantitative yield. LRMS (m/z) 402 (M+H)⁺.

Preparation of compound 35b: benzyl (2E)-3-{4-[2-(2-methyl-5-phenyl-3-furyl)ethoxy]quinolin-8-yl}prop-2-enoate

To a solution of compound 35a in toluene were added tri-(o-tolyl)phosphine (0.1 equivalent), triethylamine (2 equivalent), benzyl acrylate (3 equivalent) and palladium acetate (0.1 equivalent). The resulting mixture was heated at 90° C. for 4 hours. Concentration and silica gel chromatography provided the title compounds 35b in 40% yield. LRMS (m/z) 490 (M+H)⁺.

Preparation of compound 35a: 8-chloro-4-[2-(2-methyl-5-phenyl-3-furyl)ethoxy]quinoline

Following the procedures described in preparation of compound 28a, using 4-hydroxy-8-chloroquinoline and 2-(5-methyl-2-phenyl-1,3-oxazol-4-yl)-ethyl-4-benzenesulfonate as starting materials, the title compound 35a was produced in 70% yield. LRMS (m/z) 364 (M+H)⁺.

### OTHER EXAMPLES

Other examples of the present invention that can be prepared according to the procedures described above using the appropriate starting materials are described in Table 1 below:

| Ex. # | R¹ | Ar | A | Y | HET—Z³ | Q | T | Z |
|---|---|---|---|---|---|---|---|---|
| 36 | —CH₃ | —C₆H₆ | —O— | —CH₂—CH₂—O— | 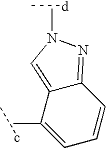 | —CH₂—CH₂—CH₂— | —(C═O)—OH—O—CH₂—CH₃ | H |
| 37 | —CH₃ | —C₆H₅ | —O— | —CH₂—CH₂—O— | 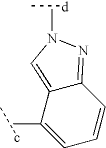 | —CH₂— | —(C═O)—OH | H |
| 38 | —CH₃ | —C₆H₅ | —O— | —CH₂—CH₂—O— | 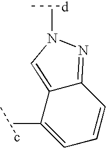 | —CH₂—CH₂—CH₂— | —(C═O)—OH | H |
| 39 | —CH₃ | —C₆H₅ | —O— | —CH₂—CH₂—CH₂—O— | 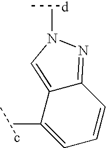 | —CH₂—CH₂—CH₂— | —(C═O)—OH | H |
| 40 | —CH₃ | —C₆H₅ | —O— | —CH₂—CH₂—CH₂—O— | 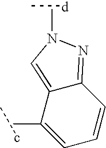 | —CH₂— | —(C═O)—OH | H |
| 41 | —CH₃ | —C₆H₅ | —O— | —CH₂—O— | 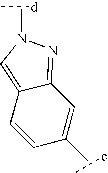 | —CH₂— | —(C═O)—OH | H |
| 42 | —C₆H₅ | —C₆H₅ | —O— | —CH₂—O— | 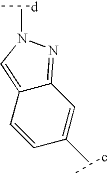 | —CH₂— | —(C═O)—OH | H |
| 43 | —C₆H₅ | —C₆H₅ | —O— | —CH₂—O— | 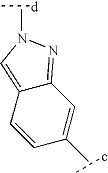 | —CH₂—CH₂— | —(C═O)—OH | H |
| 44 | —CH₃ | —C₆H₅ | —O— | —CH₂—CH₂—O— | 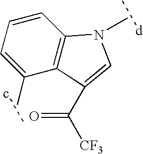 | —CH₂—CH₂— | —(C═O)—OH | H |
| 45 | —CH₃ | —C₆H₅ | —O— | —CH₂—CH₂—O— | 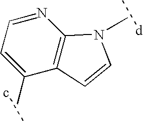 | —CH₂—CH₂— | —(C═O)—OH | H |
| 46 | —CH₃ | —C₆H₅ | —O— | —CH₂—CH₂—NH— | 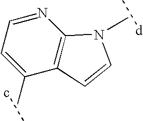 | —CH₂—CH₂— | —(C═O)—OH | H |
| 47 | —CH₃ | —C₆H₅ | —O— | —CH₂—CH₂— | 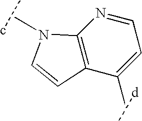 | —O—CH₂— | —(C═O)—OH | H |
| 48 | —CH₃ | —C₆H₅ | —O— | —CH₂—CH₂—O— | 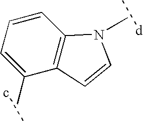 | —CH₂— | —(C═O)—OH | H |
| 49 | —CH₃ | —C₆H₅ | —O— | —CH₂—CH₂—O— | 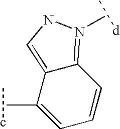 | —CH₂— | —(C═O)—OH | H |
| 50 | —CH₃ | —C₆H₅ | —O— | —CH₂—CH₂—O— | 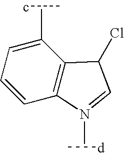 | —CH₂—CH₂— | —(C═O)—OH | H |
| 51 | —CH₃ | —C₆H₅ | —O— | —CH₂—CH₂—O— | 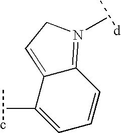 | —CH₂—CH(CO₂H)—CH₂—CH₂— | —(C═O)—OH | H |
| 52 | —CH₃ | —C₆H₅ | —O— | —CH₂—CH₂—O— | 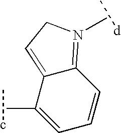 | —CH₂—CH(CO₂H)—CH₂—CH(CO₂H)—CH₂—CH₂— | —(C═O)—OH | H |
| 53 | —CH₃ | —C₆H₅ | —O— | —CH₂—CH₂—O— | 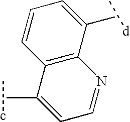 | —CH₂—CH₂— | —(C═O)—OH | H |
| 54 | —CH₃ | —C₆H₅ | —O— | —CH₂—CH₂—O— | 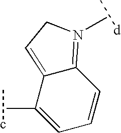 | —CH₂—CH₂—CH₂— | —(C═O)—OH | H |
| 55 | —CH₃ | —C₆H₅ | —O— | —CH₂—O— | 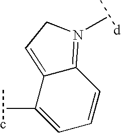 | —CH₂—CH₂—CH₂— | —(C═O)—OH | H |

### Scintillatian Proximity Assay (SPA) Assays

In the SPA assay, 3H labeled darglitazone (for PPAR-γ) or GW²³³¹ (for PPAR-α) in bound to the PPAR protein captured on SPA polylysine beads and generates radioactive count signal that can be detected by TopCounts (Packard). The PPAR-bound 3H labeled ligand can be displaced by an unlabeled compound. The Ki of the compound can be then determined by the extent of displacement at various compound concentrations.

Reagents:

SPA polylysine beads, which can be purchased from Amersham Bioscience.

3H labeled Darglitazone for PPAR-γ.

3H labeled GW2331 for PPAR-α.

PPAR proteins.

Buffer—PBS, 10% glycerol, 14 mM beta-mercaptoethanol.

Certain preferred groups of compounds possess differential selectivity toward the various PPARs. One group of preferred compounds possesses selective activity towards PPAR-γ over PPAR-α. Another preferred group of compounds possesses selective activity towards towards PPAR-γ over PPAR-δ. Another preferred group of compounds possesses selective activity towards both PPAR-α and PPAR-γ over PPAR-δ.

The compounds of the present invention with their corresponding Ki data are tabulated in the following Table 2:

TABLE 2
PPAR-γKi data

| Example # | Structure | PPAR-γKᵢ (μM) |
|---|---|---|
| 1 | 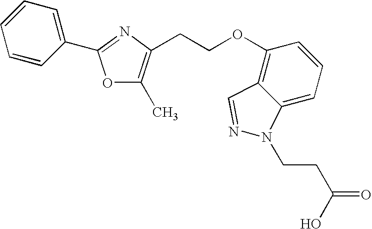 | 0.58 |
| 3 | 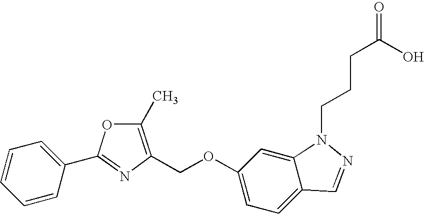 | >100 |
| 4 | 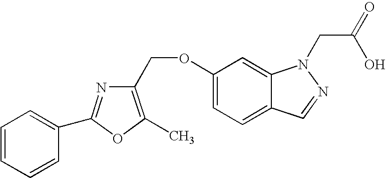 | >50 |
| 6 | 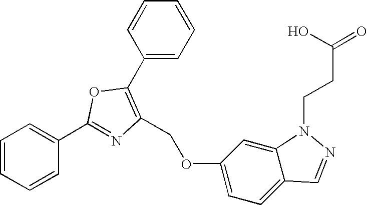 | 1.80 |
| 7 | 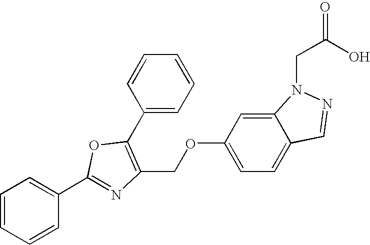 | 6.90 |
| 10 | 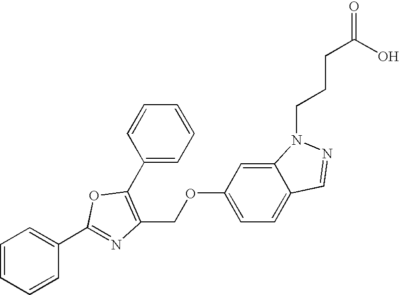 | >100 |
| 11 | 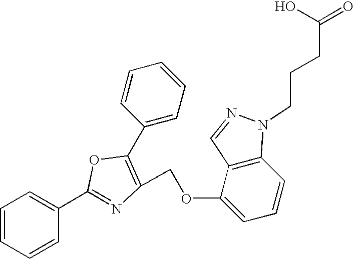 | >100 |
| 12 | 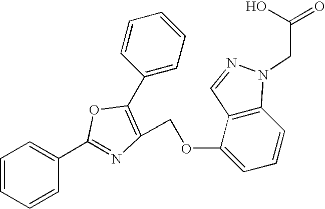 | 4.00 |
| 13 | 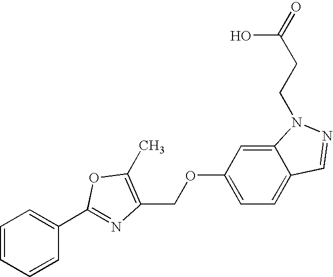 | 41.00 |
| 14 | 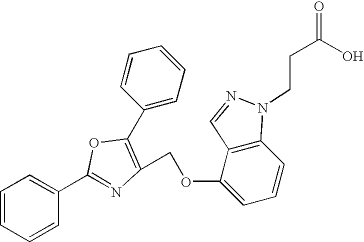 | 3.70 |
| 15 | 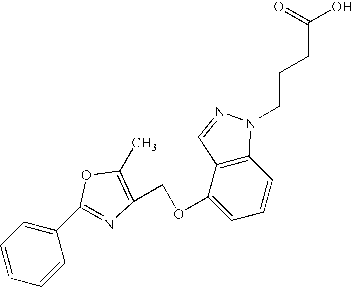 | 10.00 |
| 16 | 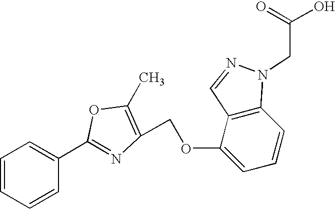 | >100 |
| 17 | 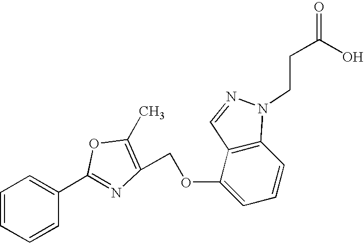 | 4.00 |
| 18 | 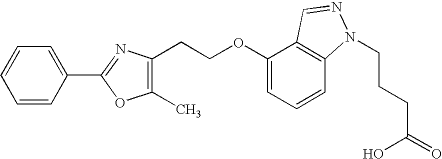 | >50 |
| 20 | 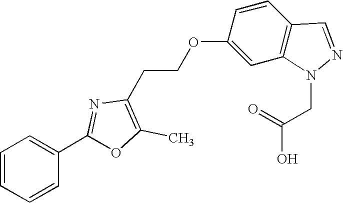 | >50 |
| 21 | 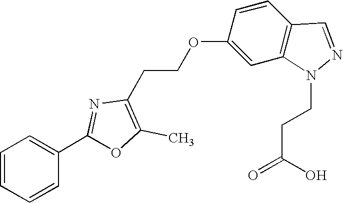 | >50 |
| 22 | 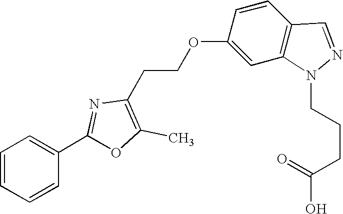 | >50 |
| 23 | 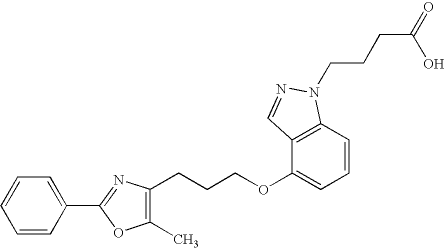 | >50 |
| 25 | 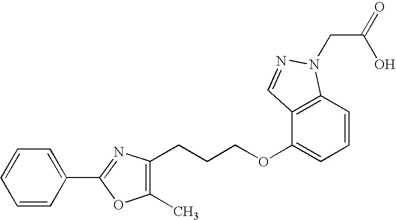 | >50 |
| 27 | 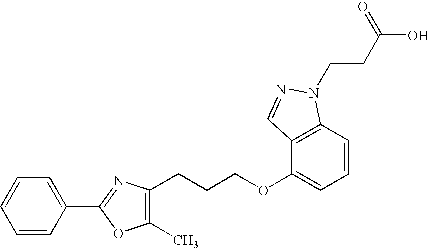 | >50 |
| 29 | 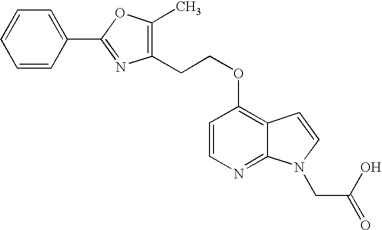 | >50 |
| 32 | 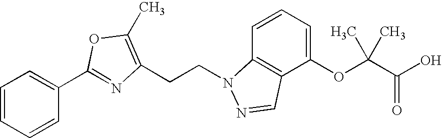 | |
| 33 | 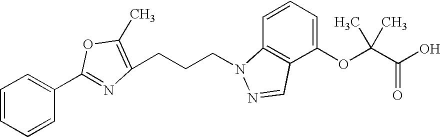 | 7.00 |
| 34 | 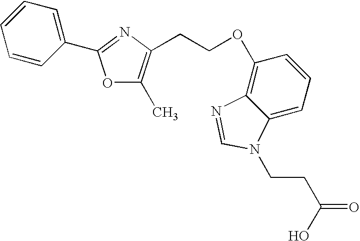 | >50 |
| 44 | 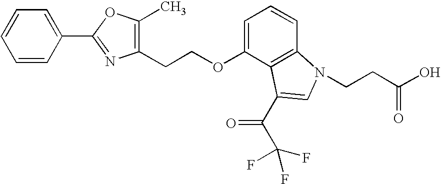 | 19.00 |
| 45 | 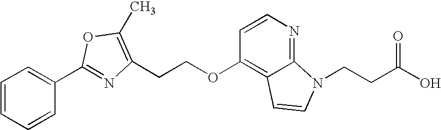 | 9.00 |
| 46 | 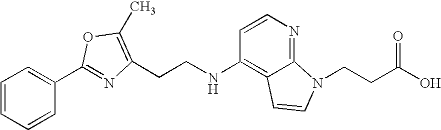 | 0.58 |
| 47 | 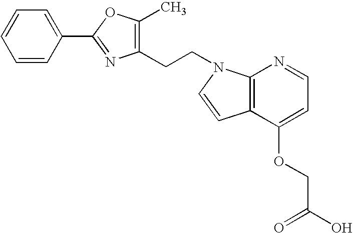 | >50 |
| 48 | 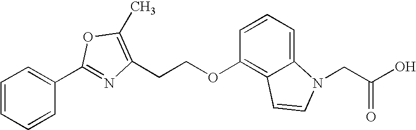 | 30.00 |
| 49 | 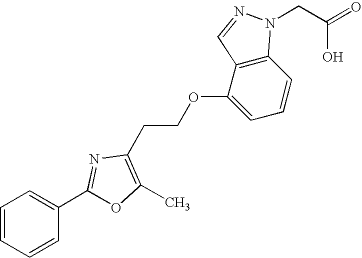 | >50 |
| 50 | 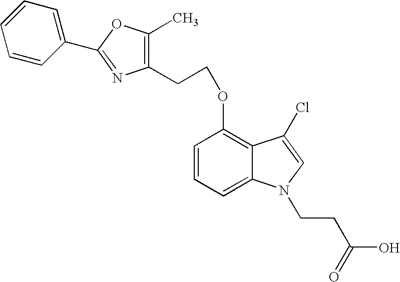 | 4.90 |
| 51 | 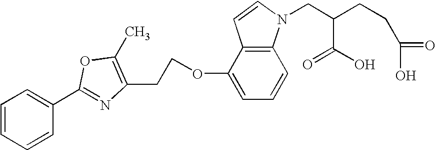 | >100 |
| 52 | 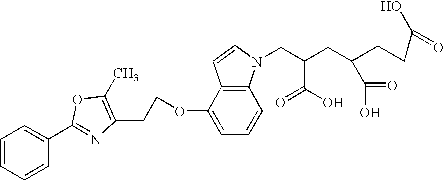 | 100.00 |
| 53 | 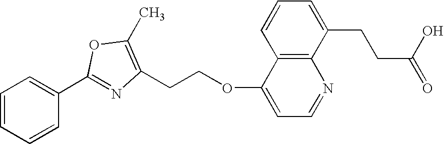 | 8.50 |
| 54 | 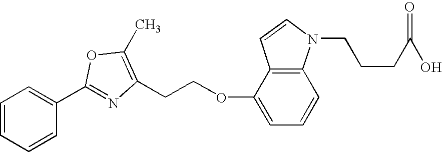 | 16.00 |
| 55 | 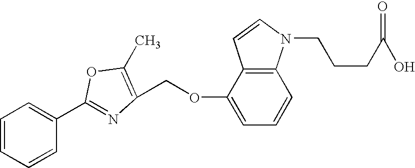 | >100 |

### Animal Tests

Fused heteroaryl compounds prepared in accordance with the above examples may be evaluated for their effect on serum glucose and serum insulin in db/db mice (C578BL/KsJ-db/db Jcl). The compounds may be dissolved in a vehicle consisting of 2% Tween80 in distilled water and administered orally. Dosage volume may be 10 ml/kg body weight. All aspects of the work including experimentation and disposal of the animals may be performed in general accordance with the International Guiding Principles for Biomedical Research Involving Animals (CIOMS Publication No. ISBN 92 90360194, 1985). Glucose-HA Assay kits (Wako, Japan) may be used for determination of serum glucose and ELISA Mouse Insulin Assay kits (SPI bio, France) may be utilized for determination of insulin. The positive control may be troglitazone (Helios Pharmaceutical, Louisville, Ky.).

The animals may be divided into twenty groups of four animals each. The animals may weigh 52+−0.5 grams at age 8-10 weeks.

Prior to any treatment a blood sample (pretreatment blood) may be taken from each animal. Four groups of animals, the vehicle groups, may receive only doses of the vehicle. Each of the vehicle groups may receive 100, 30, 10 or 1 ml/kg body weight of the vehicle orally. A solution containing compounds of the formula (I) (10 ml/kg body weight in tween 80/water) may be administered orally to the four positive control groups in doses of 100, 30, 10 and 1 ml/kg body weight respectively. The vehicle, positive control and test compound solutions may be administered to the groups immediately, 24 hours and 48 hours after drawing the pretreatment blood. Blood may be withdrawn (post treatment blood) 1.5 hours after administration of the last dose.

The serum glucose levels of the blood samples may be determined enzymatically (Mutaratose-GOD) and the insulin levels by ELISA (mouse insulin assay kit). The mean+−SEM of each group may be calculated and the percent inhibition of serum glucose and insulin may be obtained by comparison between pretreatment blood and post treatment blood. The percentage of reduction of the serum glucose and insulin levels in the post treatment blood relative to the pretreatment blood may be determined and an unpaired students t test may be applied for the comparison between the control and test solution groups and the vehicle group. A significant difference may be considered at P<0.05.

The PPAR agonist compounds of the present invention are useful in treatment conditions where modification of the effects of PPAR is of therapeutic benefit in treatment methods for mammals, including humans, involving the administration of therapeutically effective amounts of a compound of Formula (I) or a pharmaceutically acceptable salt or solvate thereof. The PPAR agonist activity of the compounds of the present invention make them particularly useful as medicaments in the treatment of PPAR mediated diseases. For example, diseases such as diabetes, both Type I and Type II, hyperglycemia, insulin resistance, obesity and certain vascular and cardiovascular diseases such as artherosclerosis and hypertension are associated with increased PPAR levels. It will be understood that the term treatment refers also to the use of the fused heteroaryl compounds of Formula (I) for the prophylaxis or prevention of PPAR mediated diseases.

The fused heteroaryl compounds of Formula (I) may be provided in suitable topical, oral and parenteral pharmaceutical formulations for use in the treatment of PPAR mediated diseases. The compounds of the present invention may be administered orally as tablets or capsules, as oily or aqueous suspensions, lozenges, troches, powders, granules, emulsions, syrups or elixars. The compositions for oral use may include one or more agents for flavoring, sweetening, coloring and preserving in order to produce pharmaceutically elegant and palatable preparations. Tablets may contain pharmaceutically acceptable excipients as an aid in the manufacture of such tablets. As is conventional in the art these tablets may be coated with a pharmaceutically acceptable enteric coating, such as glyceryl monostearate or glyceryl distearate, to delay disintegration and absorption in the gastrointestinal tract to provide a sustained action over a longer period.

Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain active ingredients in admixture with excipients suitable for the manufacture of an aqueous suspension. Such excipients may be a suspending agent, such as sodium carboxymethyl cellulose, methyl cellulose, hydroxypropylmethyl cellulose, sodium alginate, polyvinylpyrrolidone, gum tragacanth and gum acacia; a dispersing or wetting agent that may be a naturally occuring phosphatide such as lecithin, a condensation product of ethylene oxide and a long chain fatty acid, for example polyoxyethylene stearate, a condensation product of ethylene oxide and a long chain aliphatic alcohol such as heptadecaethylenoxycetanol, a condensation product of ethylene oxide and a partial ester derived from a fatty acid and hexitol such as polyoxyethylene sorbitol monooleate or a fatty acid hexitol anhydrides such as polyoxyethylene sorbitan monooleate.

The pharmaceutical compositions may be in the form of a sterile injectable aqueous or oleagenous suspension. This suspension may be formulated according to know methods using those suitable dispersing or wetting agents and suspending agents that have been mentioned above. The sterile injectable preparation may also be formulated as a suspension in a non toxic perenterally-acceptable diluent or solvent, for example as a solution in 1,3-butanediol. Among the accetable vehicles and solvents that may be employed are water, Ringers solution and isotonic sodium chloride solution. For this purpose any bland fixed oil may be employed including synthetic mono- or diglycerides. In addition fatty acids such as oleic acid find use in the preparation of injectables.

The fused heteroaryl compounds of Formula (I) may also be administered in the form of suppositories for rectal administration of the drug. These compositions can be prepared by mixing the drug with a suitable non-irritating excipient that is solid at about room temperature but liquid at rectal temperature and will therefore melt in the rectum to release the drug. Such materials include cocoa butter and other glycerides.

For topical use preparations, for example, creams, ointments, jellies solutions, or suspensions, containing the compounds of the present invention are employed.

The fused heteroaryl compounds of Formula (I) may also be administered in the form of liposome delivery systems such as small unilamellar vesicles, large unilamellar vesicles and multimellar vesicles. Liposomes can be formed from a variety of phospholipides, such as cholesterol, stearylamine or phosphatidylcholines.

Dosage levels of the compounds of the present invention are of the order of about 0.5 mg/kg body weight to about 100 mg/kg body weight. A preferred dosage rate is between about 30 mg/kg body weight to about 100 mg/kg body weight. It will be understood, however, that the specific dose level for any particular patient will depend upon a number of factors including the activity of the particular compound being administered, the age, body weight, general health, sex, diet, time of administration, route of administration, rate of excretion, drug combination and the severity of the particular disease undergoing therapy. To enhance the therapeutic activity of the present compounds they may be administered concomitantly with other orally active antidiabetic compounds such as the sulfonylureas, for example, tolbutamide and the like.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

What is claimed:

1. A compound of formula (I): 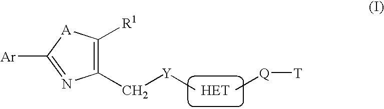
wherein:
Ar is (C₃-C₁₀)cycloalkyl, (C₂-C₁₀)heterocyclyl, (C₆-C₁₀)aryl, or (C₁-C₁₀)heteroaryl, wherein each Ar is optionally substituted with one to four substituents selected from Z;
A is —CH₂—, —NH, —O—, or —S—;
R¹ is (C₁-C₈)alkyl, (C₃-C₁₀)cycloalkyl, (C₂-C₁₀)heterocyclyl, (C₆-C₁₀)aryl, or (C₁-C₁₀)heteroaryl; wherein each R¹ is optionally substituted with one to four substituents selected from Z¹;
Y is selected from the group consisting of —(CH₂)ₙ—, —(CH₂)ₙ—NR¹⁵—, —(CH₂)ₙ—O—, and —(CH₂)ₙ—S—; wherein each n is independently 0, 1, 2, or 3;
and R¹⁵ is hydrogen, (C₁-C₈)alkyl, (C₃-C₁₀)cycloalkyl, (C₂-C₁₀)heterocyclyl, (C₆-C₁₀)aryl, or (C₁-C₁₀)heteroaryl; wherein each R¹⁵ is optionally substituted with one to four substituents selected from Z²;
Q is selected from the group consisting of —(CR²R³)ₘ—, —(CR²R³)ₘ—N¹⁵—, —N¹⁵—(CR²R³)ₘ—, (CR²R³)ₘ—O—, —O—(CR²R³)ₘ—, —S—(CR²R³)ₘ—, and —(CR²R³)ₘ—S—; wherein each m is independently 1, 2, 3, 4, 5, or 6; 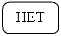
is a fused (C₆-C₁₂)heteroaryl optionally substituted one to four substituents selected from Z³, wherein Z³ may be in any ring of the fused (C₆-C₁₂)heteroaryl, having the formula: 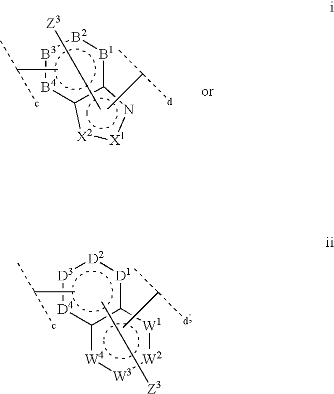
wherein the dotted lines are optional double bonds such that said fused (C₆-C₁₂)heteroaryl is aromatic;
Each of X¹, X², W¹, W², W³, W⁴, B¹, B², B³, B⁴, D¹, D², D³ and D⁴ are independently ═CH— or ═N—;
At least one of X¹, X², B¹, B², B³, and B⁴ must be ═N—;
At least one of W¹, W², W³, W⁴, D¹, D², D³ and D⁴ must be ═N—;
Wherein each --c is a point of attachment to the group —Y— and each ---d is a point of attachment to the group —Q—;
Each of Z, Z¹, Z², and Z³ are selected from the group consisting of:
(c) H, F, Cl, Br, I, CF₃, or CN;
(d) (C₁-C₈)alkyl optionally substituted with one to four substituents independently selected from R⁷;
(c) —C(═O)—R⁴ {wherein R⁴ is selected from the group consisting of H, OH, CF₃, (C₁-C₈)alkyl, (C₁-C₈)alkyl-O—, (C₃-C₁₀)cycloalkyl, (C₃-C₁₀)cycloalkyl-O—; (C₂-C₁₀)heterocyclyl, (C₂-C₁₀)heterocyclyl-O—; (C₆-C₁₀)aryl, (C₆-C₁₀)aryl-O—, (C₁-C₁₀)heteroaryl, and (C₁-C₁₀)heteroaryl-O—};
(d) —C(═O)—NR⁵R⁶ {wherein R⁵ is H or (C₁-C₈)alkyl; and wherein R⁶ is selected from the group consisting of H, (C₁-C₈)alkyl, —CH₂—(C═O)—O—(C₁-C₈)alkyl, (C₃-C₁₀)cycloalkyl, (C₂-C₁₀)heterocyclyl, (C₆-C₁₀)aryl, and (C₁-C₁₀)heteroaryl};
(j) (C₃-C₁₀)cycloalkyl, (C₂-C₁₀)heterocyclyl, (C₆-C₁₀)aryl, or (C₁-C₁₀)heteroaryl;
(k) NR⁹R¹⁰ {wherein R⁹ is independently H or (C₁-C₈)alkyl; R¹⁰ is selected from the group consisting of —C(═O)—O—C(CH₃)₃, (C₁-C₈)alkyl, (C₃-C₁₀)cycloalkyl, (C₂-C₁₀)heterocyclyl, (C₆-C₁₀)aryl, and (C₁-C₁₀)heteroaryl; or R⁹ and R¹⁰ may optionally be taken together with the nitrogen to which they are attached to form a 5 to 8-membered heteroaryl or heterocyclyl ring};
(l) R¹¹—O— {wherein R¹¹ is selected from the group consisting of (C₁-C₈)alkyl, (C₃-C₁₀)cycloalkyl, (C₂-C₁₀)heterocyclyl, (C₆-C₁₀)aryl, and (C₁-C₁₀)heteroaryl};
(m) R¹²—SOₚ— {wherein R¹² is selected from the group consisting of (C₁-C₈)alkyl, (C₃-C₁₀)cycloalkyl, (C₂-C₁₀)heterocyclyl, (C₆-C₁₀)aryl, and (C₁-C₁₀)heteroaryl; and wherein p is 0, 1, or 2}; and
(n) R¹³R¹⁴N—SO_{q}— {wherein R¹³ is H or (C₁-C₈)alkyl; R¹⁴ is (C₁-C₈)alkyl, (C₃-C₁₀)cycloalkyl, (C₂-C₁₀)heterocyclyl, (C₆-C₁₀)aryl, or (C₁-C₁₀)heteroaryl; or R¹³ and R¹⁴ may optionally be taken together with the nitrogen to which they are attached to form a 5 to 8-membered heteroaryl or heterocyclyl ring; and wherein q is 1 or 2};
Each of R² and R³ is independently (a) H; (b) (C₁-C₈)alkyl optionally substituted with one to four substituents independently selected from R⁷; (c) COOH; or (d) (C₆-C₁₀)aryl optionally substituted with one to four substituents independently selected from R⁸;
Wherein each of R⁷ and R⁸ are independently selected from the group consisting of:
(g) F, Cl, Br, I, CN, CF₃, or NO₂;
(h) —NR⁹R¹⁰ {wherein R⁹ is independently H or (C₁-C₈)alkyl; R¹⁰ is selected from the group consisting of —C(═O)—O—C(CH₃)₃, (C₁-C₈)alkyl, (C₃-C₁₀)cycloalkyl, (C₂-C₁₀)heterocyclyl, (C₆-C₁₀)aryl, and (C₁-C₁₀)heteroaryl; or R⁹ and R¹⁰ may optionally be taken together with the nitrogen to which they are attached to form a 5 to 8-membered heteroaryl or heterocyclyl ring};
(i) R¹¹—O— {wherein R¹¹ is selected from the group consisting of (C₁-C₈)alkyl, (C₃-C₁₀)cycloalkyl, (C₂-C₁₀)heterocyclyl, (C₆-C₁₀)aryl, and (C₁-C₁₀)heteroaryl};
(j) R¹²—SOₚ— {wherein R¹² is selected from the group consisting of (C₁-C₈)alkyl, (C₃-C₁₀)cycloalkyl, (C₂-C₁₀)heterocyclyl, (C₆-C₁₀)aryl, and (C₁-C₁₀)heteroaryl; and wherein p is 0, 1, or 2}; and
(k) R¹³R¹⁴N—SO_{q}— {wherein R¹³ is H or (C₁-C₈)alkyl; R¹⁴ is (C₁-C₈)alkyl, (C₃-C₁₀)cycloalkyl, (C₂-C₁₀)heterocyclyl, (C₆-C₁₀)aryl, or (C₁-C₁₀)heteroaryl; or R¹³ and R¹⁴ may optionally be taken together with the nitrogen to which they are attached to form a 5 to 8-membered heteroaryl or heterocyclyl ring; and wherein q is 1 or 2};
(l) T is selected from the group consisting of —(C═O)—OH, —(C═O)—OR¹⁵, —(C═O)—OM {wherein M is an alkali metal or alkaline earth metal}, tetrazolyl, thiazolidinyl, —SO₂—NH—R¹⁵, —NH—SO₂—R¹⁵, —(C═O)—NH—SO₂—R¹⁵, and other acid prodrug or isosteres thereof;
or a pharmaceutically acceptable salt thereof.

2. The compound according to claim 1 wherein 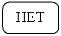
is a fused (C₆-C₁₂)heteroaryl optionally substituted one to four substituents selected from Z³, wherein Z³ may be in any ring of the fused (C₆-C₁₂)heteroaryl, having the formula: 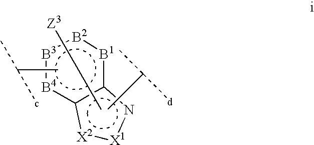
selected from the group consisting of:

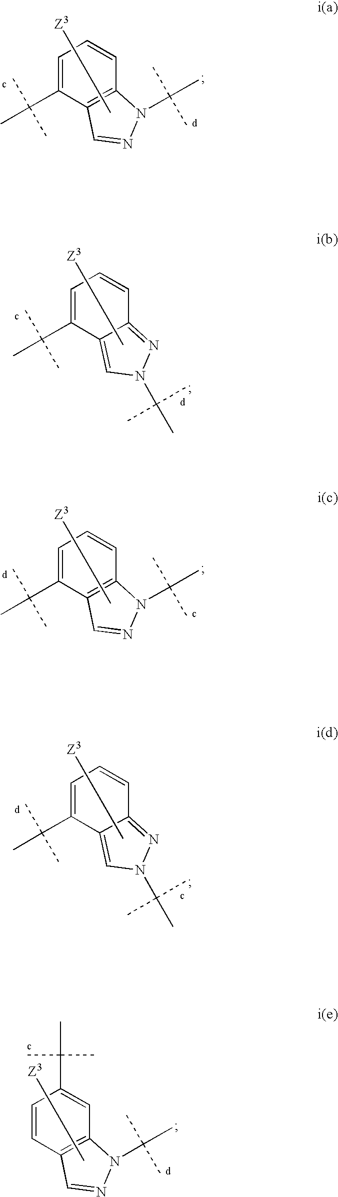

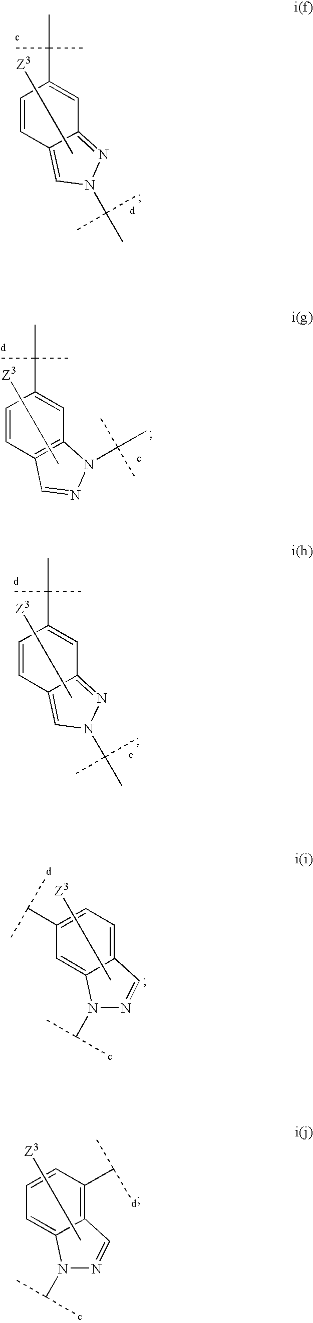

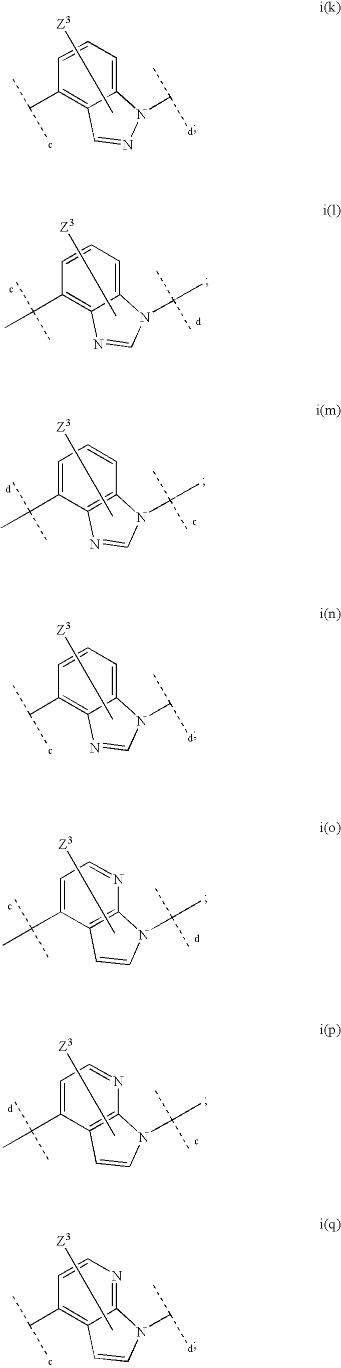

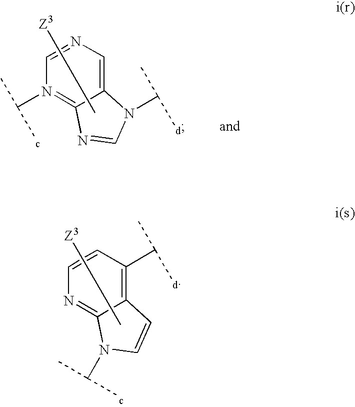

3. The compound according to claim 1 wherein 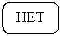
is a fused (C₆-C₁₂)heteroaryl optionally substituted one to four substituents selected from Z³, wherein Z³ may be in any ring of the fused (C₆-C₁₂)heteroaryl, having the formula: 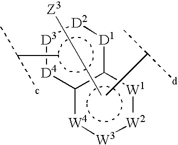
selected from the group consisting of:

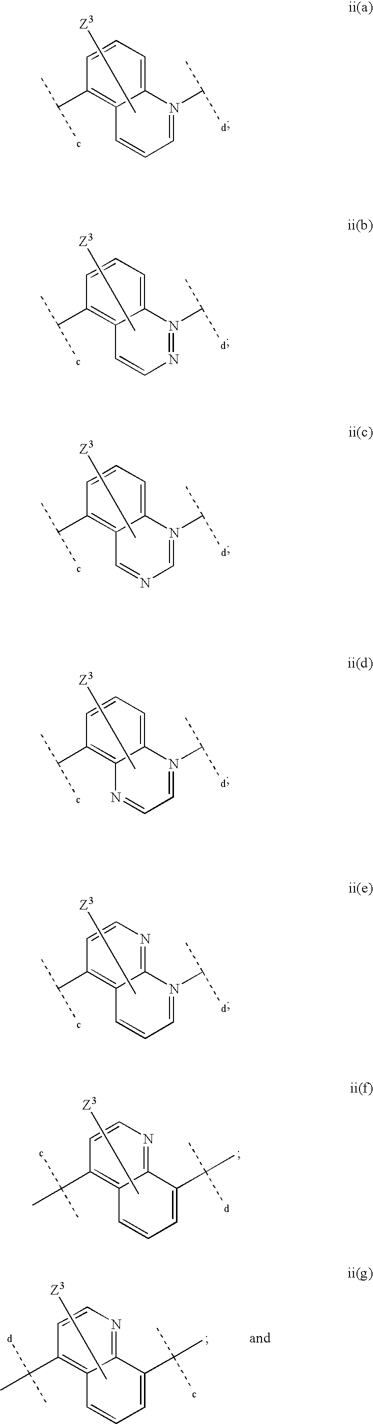

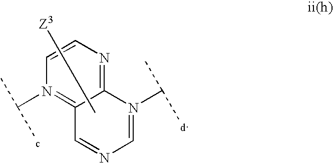

4. The compound according to claim 1 wherein said Ar is phenyl.

5. The compound according to claim 1 wherein said A is —O—.

6. The compound according to claim 1 wherein said R¹ is (C₁-C₈)alkyl.

7. The compound according to claim 1 wherein said R¹ is (C₆-C₁₀)aryl.

8. The compound according to claim 1 wherein said Q is —(CR²R³)ₘ—, m is 2 or 3, and each of R² and R³ is hydrogen or (C₁-C₈)alkyl.

9. The compound according to claim 1 wherein said Q is —(CR²R³)ₘ—NH—, m is 1 or 2, and each of R² and R³ is hydrogen or unsubstituted (C₁-C₈)alkyl.

10. The compound according to claim 1 wherein said Q is —(CR²R³)ₘ—O—, m is 1 or 2, and each of R² and R³ is hydrogen or unsubstituted (C₁-C₈)alkyl.

11. The compound according to claim 1 wherein said Q is —(CR²R³)ₘ—S—, m is 1 or 2, and each of R² and R³ is hydrogen or unsubstituted (C₁-C₈)alkyl.

12. The compound according to claim 1 wherein said T is —(C═O)—OH.

13. The compound according to claim 1 wherein said T is selected from the group consisting of tetrazolyl, thiazolidinyl, —SO₂—NH—R¹⁵, —NH—SO₂—R¹⁵, —(C═O)—NH—SO₂—R¹⁵, and other acid prodrug or isosteres thereof.

14. The compound according to claim 1 wherein said Z³ is selected from the group consisting of F, Cl, Br, or I.

15. The compound according to claim 1 wherein said Z³ is (C₁-C₈)alkyl.

16. The compound according to claim 1 wherein said Y is —(CH₂)ₙ—O— and n is 1, 2, or 3.

17. The compound according to claim 1 wherein said Y is —(CH₂)ₙ—NR¹⁵—, wherein R¹⁵ is hydrogen, (C₁-C₈)alkyl or (C₃-C₁₀)cycloalkyl, and n is 1, 2, or 3.

18. The compound according to claim 1 wherein said Y is —(CH₂)ₙ— and n is 1, 2, or 3.

19. The compound according to claim 1 wherein said Y is —(CH₂)ₙ—S— and n is 1, 2, or 3.

20. The compound according to claim 1 selected from the group consisting of: 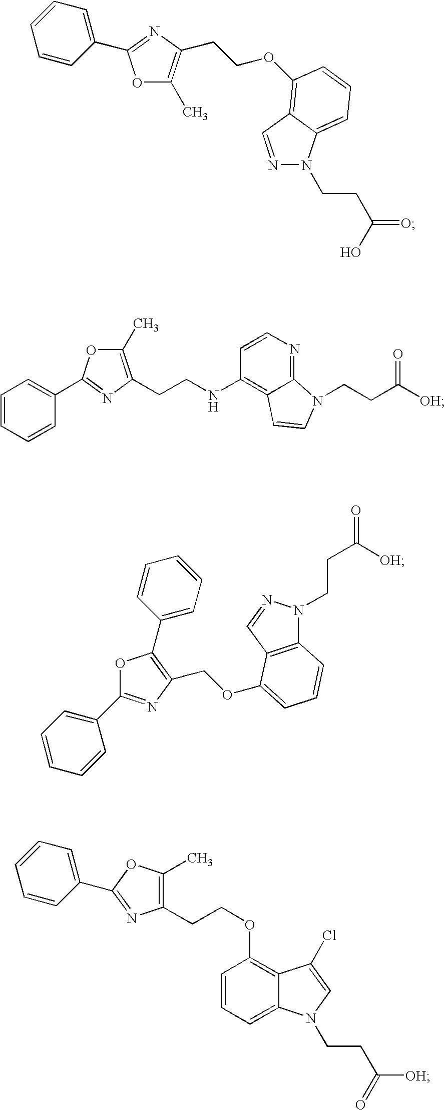 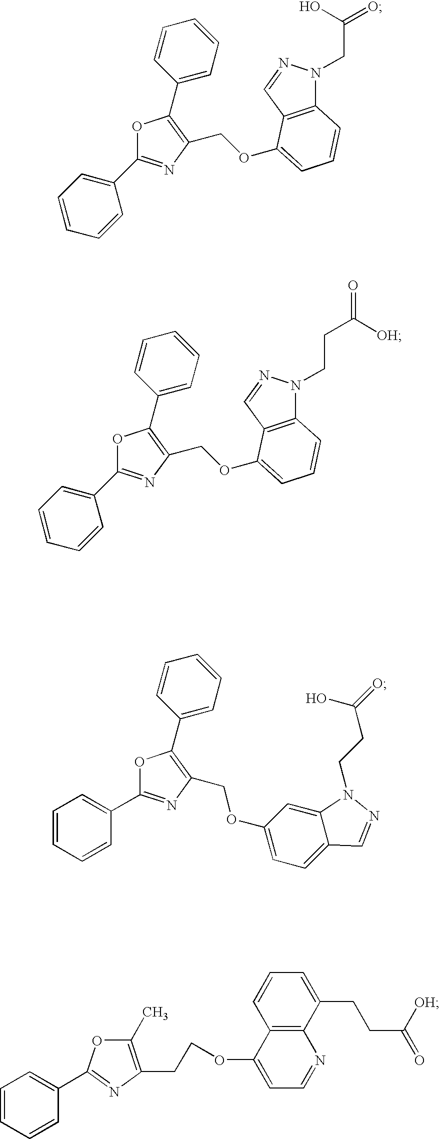 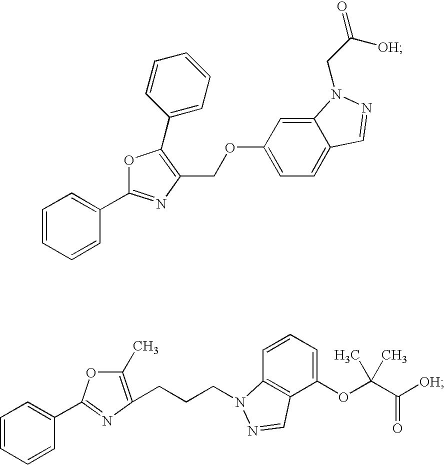
and the pharmaceutically acceptable salts thereof.

21. A method of treating non-insulin dependent diabetes mellitus in a mammal comprising administering to the mammal in need thereof a therapeutically effective amount of a fused heteroaryl compound according to claim 1.

22. The method of treating non-insulin dependent diabetes mellitus according to claim 21 wherein said mammal has an impaired glucose tolerance.

23. A method of treating polycystic ovarian syndrome in a mammal comprising administering to the mammal in need thereof a therapeutically effective amount of a fused heteroaryl compound according to claim 1.

24. A method of treating obesity in a mammal comprising administering to the mammal in need thereof a therapeutically effective amount of a fused heteroaryl compound according to claim 1.

25. A method of reducing body weight in an obese mammal comprising administering to the mammal in need thereof a therapeutically effective amount of a fused heteroaryl compound according to claim 1.

26. A method of treating hyperglycemia in a mammal comprising administering to the mammal in need thereof a therapeutically effective amount of a fused heteroaryl compound according to claim 1.

27. A method of treating hyperlipidemia in a mammal comprising administering to the mammal in need thereof a therapeutically effective amount of a fused heteroaryl compound according to claim 1.

28. A method of treating hypercholesteremia in a mammal comprising administering to the mammal in need thereof a therapeutically effective amount of a fused heteroaryl compound according to claim 1.

29. A method of treating atherosclerosis in a mammal comprising administering to the mammal in need thereof a therapeutically effective amount of a fused heteroaryl compound according to claim 1.

30. A method of treating hypertriglyceridemia in a mammal comprising administering to the mammal in need thereof a therapeutically effective amount of a fused heteroaryl compound according to claim 1.

31. A method of treating hyperinsulinemia in a mammal comprising administering to the mammal in need thereof a therapeutically effective amount of a fused heteroaryl compound according to claim 1.

32. A method of treating a patient suffering from abnormal insulin and/or evidence of glucose disorders associated with circulating glucocorticoids, growth hormone, catecholamines, glucagon, or parathyroid hormone, comprising administering to said patient a therapeutically effective amount of a fused heteroaryl compound according to claim 1.

33. A method of treating insulin resistance syndrome in humans comprising administering to a patient in need of treatment a therapeutically effective amount of a fused heteroaryl compound according to claim 1.

34. A method of treating PPAR-related disorders in humans comprising administering to a patient in need of treatment a therapeutically effective amount of a fused heteroaryl compound according to claim 1.

35. A method of modulating PPAR activity in a mammal, comprising administering to a mammal a therapeutically effective amount of a fused heteroaryl compound according to claim 1.

36. A method of lowering blood glucose in a mammal, comprising administering to a mammal an amount of a fused heteroaryl compound according to claim 1 effective to lower blood glucose levels.

37. A method of modulating fat cell differentiation in a mammal, comprising administering to a mammal a therapeutically effective amount of a fused heteroaryl compound according to claim 1.

38. A method of modulating processes mediated by PPAR in a mammal, comprising administering to a mammal a therapeutically effective amount of a fused heteroaryl compound according to claim 1.

39. The method of modulating processes mediated by PPAR in a mammal according to claim 39, wherein said fused heteroaryl compound according to claim 1 is administered to said mammal in combination with at least one compound selected from the group consisting of α-glucosidase inhibitors, aldose reductase inhibitors, biguanide preparations, statin base compounds, squalene synthesis inhibitors, fibrate base compounds, LDL catabolism promoters and angiotensin-converting enzyme inhibitors.

40. A method of increasing insulin sensitivity in mammals, comprising administering to a mammal a therapeutically effective amount of a fused heteroaryl compound according to claim 1.

41. A composition comprising at least one modulator of PPAR according to claim 1 and a pharmaceutically acceptable carrier thereof.

42. A composition according to claim 40 further comprising at least one compound selected from the group consisting of α-glucosidase inhibitors, aldose reductase inhibitors, biguanide preparations, statin base compounds, squalene synthesis inhibitors, fibrate base compounds, LDL catabolism promoters and angiotensin-converting enzyme inhibitors.
